(19) United States
(12) Patent Application Publication
(10) Pub. No.: US 2025/0121154 A1
(43) Pub. Date: Apr. 17, 2025

(54) BED DEVICE SYSTEM AND METHODS

(71) Applicant: Eight Sleep Inc., New York, NY (US)

(72) Inventors: Matteo FRANCESCHETTI, Miami Shores, FL (US); Massimo ANDREASI BASSI, Union City, NJ (US)

(21) Appl. No.: 18/990,538

(22) Filed: Dec. 20, 2024

Related U.S. Application Data
(63) Continuation of application No. 18/741,237, filed on Jun. 12, 2024.
Application No. 18/741,237 is a continuation of application No. 17/526,074, filed on Nov. 15, 2021, now Pat. No. 12,053,591.
Application No. 17/526,074 is a continuation of application No. 17/225,487, filed on Apr. 8, 2021, now abandoned.
Application No. 17/225,487 is a continuation of application No. 17/001,799, filed on Aug. 25, 2020, now abandoned.
Application No. 17/001,799 is a continuation of application No. 16/457,306, filed on Jun. 28, 2019, now Pat. No. 10,792,461.
Application No. 16/457,306 is a continuation of application No. 16/148,376, filed on Oct. 1, 2018, now abandoned.
Application No. 16/148,376 is a continuation of application No. 15/602,969, filed on May 23, 2017, now abandoned.
Application No. 15/602,969 is a continuation of application No. 14/732,624, filed on Jun. 5, 2015, now Pat. No. 9,981,107.
(63) Continuation of application No. 17/977,170, filed on Oct. 31, 2022.
Application No. 17/977,170 is a continuation of application No. 17/704,417, filed on Mar. 25, 2022, now abandoned.
Application No. 17/704,417 is a continuation of application No. 17/405,302, filed on Aug. 18, 2021, now abandoned.
Application No. 17/405,302 is a continuation of application No. 17/144,619, filed on Jan. 8, 2021, now abandoned.
Application No. 17/144,619 is a continuation of application No. 16/886,050, filed on May 28, 2020, now abandoned.
Application No. 16/886,050 is a continuation of application No. 16/589,808, filed on Oct. 1, 2019, now abandoned.
Application No. 16/589,808 is a continuation-in-part of application No. 15/449,739, filed on Mar. 3, 2017, now abandoned.
Application No. 15/449,739 is a continuation of application No. 14/969,932, filed on Dec. 15, 2015, now Pat. No. 9,586,021.
Application No. 14/969,932 is a continuation-in-part of application No. 14/946,496, filed on Nov. 19, 2015, now abandoned.
Application No. 14/946,496 is a continuation-in-part of application No. 14/732,646, filed on Jun. 5, 2015, now abandoned.
(63) Continuation of application No. 17/982,838, filed on Nov. 8, 2022.
Application No. 17/982,838 is a continuation of application No. 17/726,756, filed on Apr. 22, 2022, now abandoned.
Application No. 17/726,756 is a continuation of application No. 17/470,312, filed on Sep. 9, 2021, now abandoned.
Application No. 17/470,312 is a continuation of application No. 17/009,189, filed on Sep. 1, 2020, now abandoned.
Application No. 17/009,189 is a continuation of application No. 16/732,750, filed on Jan. 2, 2020, now abandoned.
Application No. 16/732,750 is a continuation of application No. 16/422,785, filed on May 24, 2019, now abandoned.
Application No. 16/422,785 is a continuation of application No. 16/148,307, filed on Oct. 1, 2018, now abandoned.
Application No. 16/148,307 is a continuation of application No. 14/969,902, filed on Dec. 15, 2015, now abandoned.
Application No. 14/969,902 is a continuation-in-part of application No. 14/732,646, filed on Jun. 5, 2015, now abandoned.
(63) Continuation of application No. 17/982,843, filed on Nov. 8, 2022.
Application No. 17/982,843 is a continuation of application No. 17/879,341, filed on Aug. 2, 2022, now abandoned.
Application No. 17/879,341 is a continuation of application No. 17/556,402, filed on Dec. 20, 2021, now abandoned.
Application No. 17/556,402 is a continuation of application No. 17/320,626, filed on May 14, 2021, now abandoned.
Application No. 17/320,626 is a continuation of application No. 17/070,324, filed on Oct. 14, 2020, now abandoned.
Application No. 17/070,324 is a continuation of application No. 16/810,435, filed on Mar. 5, 2020, now abandoned.
Application No. 16/810,435 is a continuation of application No. 16/451,755, filed on Jun. 25, 2019, now abandoned.
Application No. 16/451,755 is a continuation of application No. 15/293,049, filed on Oct. 13, 2016, now abandoned.
Application No. 15/293,049 is a continuation of application No. 15/178,124, filed on Jun. 9, 2016, now Pat. No. 9,694,156.
Application No. 15/178,124 is a continuation-in-part of application No. 14/732,646, filed on Jun. 5, 2015, now abandoned.
(60) Provisional application No. 62/008,480, filed on Jun. 5, 2014.
(60) Provisional application No. 62/008,480, filed on Jun. 5, 2014.
(60) Provisional application No. 62/024,945, filed on Jul. 15, 2014.
(60) Provisional application No. 62/159,177, filed on May 8, 2015.
(60) Provisional application No. 62/161,142, filed on May 13, 2015.

(51) Int. Cl. A61M 21/00; A47C 21/00; A47C 21/04; A47C 31/12; A47G 9/02; A61B 5/00; A61B 5/01; A61B 5/0205; A61B 5/024; A61B 5/08; A61B 5/11; A61B 5/117; A61M 16/16; A61M 21/02; G05B 19/042; G05D 23/13; G08B 6/00; G16H 40/67; H05B 1/02

(52) U.S. Cl. CPC A61M 21/00; A47C 21/00; A47C 21/044; A47C 21/048; A47C 31/123; A47G 9/0238; A61B 5/0022; A61B 5/01; A61B 5/02055; A61B 5/024; A61B 5/0816; A61B 5/11; A61B 5/117; A61B 5/4266; A61B 5/4812; A61B 5/6892; A61B 5/7455; A61M 21/02; G05B 19/0428; G05D 23/1393; G08B 6/00; G16H 40/67; H05B 1/0272; A61B 2505/07; A61B 2560/0242; A61B 2560/0475; A61B 2562/0271; A61B 2562/063; A61M 16/161; A61M 2021/0022; A61M 2021/0066; A61M 2021/0083; A61M 2205/0294; A61M 2205/18; A61M 2205/3303; A61M 2205/3306; A61M 2205/3368; A61M 2205/3375; A61M 2205/3584; A61M 2205/3592; A61M 2205/3673; A61M 2205/50; A61M 2205/505; A61M 2230/06; A61M 2230/42; A61M 2230/50; A61M 2230/63; G05B 2219/2614

(57) ABSTRACT

Introduced are a bed device system and methods for: gathering human biological signals, such as heart rate, breathing rate, or temperature; analyzing the gathered human biological signals; and controlling the bed device system, e.g., a temperature of the bed device, based on the analysis.

### CROSS-REFERENCE TO RELATED APPLICATIONS

This application is a continuation of U.S. application Ser. No. 18/741,237, filed Jun. 12, 2024, which is a continuation of U.S. application Ser. No. 17/526,074, filed Nov. 15, 2021, now U.S. Pat. No. 12,053,591, issued Aug. 6, 2024, which is a continuation of U.S. application Ser. No. 17/225,487, filed Apr. 8, 2021, which is a continuation of U.S. application Ser. No. 17/001,799, filed Aug. 25, 2020, which is a continuation of U.S. application Ser. No. 16/457,306, filed Jun. 28, 2019, now U.S. Pat. No. 10,792,461, issued Oct. 6, 2020, which is a continuation of U.S. application Ser. No. 16/148,376, filed Oct. 1, 2018, which is a continuation of U.S. application Ser. No. 15/602,969, filed May 23, 2017, which is a continuation of U.S. application Ser. No. 14/732,624, filed Jun. 5, 2015, now U.S. Pat. No. 9,981,107, issued May 29, 2018, each of which is entirely incorporated herein by reference. The U.S. application Ser. No. 14/732,624 claims priority to the following U.S. provisional patent applications: U.S. Provisional Patent Application No. 62/008,480, filed Jun. 5, 2014; U.S. Provisional Patent Application No. 62/024,945, filed Jul. 15, 2014; U.S. Provisional Patent Application No. 62/159,177, filed May 8, 2015; and U.S. Provisional Patent Application No. 62/161,142, filed May 13, 2015, each of which is entirely incorporated herein by reference.

This application is also a continuation of U.S. application Ser. No. 17/977,170, filed Oct. 31, 2022, which is a continuation of U.S. application Ser. No. 17/704,417, filed Mar. 25, 2022, which is a continuation of U.S. application Ser. No. 17/405,302, filed Aug. 18, 2021, which is a continuation of U.S. application Ser. No. 17/144,619, filed Jan. 8, 2021, which is a continuation of U.S. Application No. 16,886,050, filed May 28, 2020, which is a continuation of U.S. application Ser. No. 16/589,808, filed Oct. 1, 2019, which is a continuation-in-part of U.S. application Ser. No. 15/449,739, filed Mar. 3, 2017, which is a continuation of U.S. application Ser. No. 14/969,932, filed Nov. 19, 2015, Now U.S. Pat. No. 9,586,021, issued Mar. 7, 2017, which is a continuation-in-part of U.S. application Ser. No. 14/946,496, filed Nov. 19, 2015, which is a continuation-in-part of U.S. application Ser. No. 14/732,646, filed Jun. 5, 2015, each of which is entirely incorporated herein by reference. The U.S. application Ser. No. 14/732,646 claims priority to the following U.S. provisional patent applications: U.S. Provisional Patent Application No. 62/008,480, filed Jun. 5, 2014; U.S. Provisional Patent Application No. 62/024,945, filed Jul. 15, 2014; U.S. Provisional Patent Application No. 62/159,177, filed May 8, 2015; and U.S. Provisional Patent Application No. 62/161,142, filed May 13, 2015, each of which is entirely incorporated herein by reference.

This application is also a continuation of U.S. application Ser. No. 17/982,838, filed Nov. 8, 2022, which is a continuation of U.S. application Ser. No. 17/726,756, filed Apr. 22, 2022, which is a continuation of U.S. patent application Ser. No. 17/470,312, filed Sep. 9, 2021, which is a continuation of U.S. patent application Ser. No. 17/009,189, filed Sep. 1, 2020, which is a continuation of U.S. patent application Ser. No. 16/732,750, filed Jan. 2, 2020, which is a continuation of U.S. patent application Ser. No. 16/422,785, filed May 24, 2019, which is a continuation of U.S. patent application Ser. No. 16/148,307, filed Oct. 1, 2018, which is a continuation of U.S. patent application Ser. No. 14/969,902, filed on Dec. 15, 2015, which is a continuation-in-part of U.S. patent application Ser. No. 14/732,646, filed on Jun. 5, 2015, each of which is entirely incorporated herein by reference.

This application is also a continuation of U.S. Ser. No. 17/982,843, filed Nov. 8, 2022, which is a continuation of U.S. application Ser. No. 17/879,341, filed Aug. 2, 2022, which is a continuation of U.S. application Ser. No. 17/556,402, filed Dec. 20, 2021, which is a continuation of U.S. application Ser. No. 17/320,626, Filed May 14, 2021, which is a continuation of U.S. application Ser. No. 17/070,324, filed Oct. 14, 2020, which is a continuation of U.S. application Ser. No. 16/810,435, filed Mar. 5, 2020, which is a continuation of U.S. patent application Ser. No. 16/451,755, filed Jun. 25, 2019, which is a continuation of U.S. patent application Ser. No. 15/293,049, filed Oct. 13, 2016, which is a continuation of U.S. patent application Ser. No. 15/178,124, filed Jun. 9, 2016, now U.S. Pat. No. 9,694,156, issued on Jul. 4, 2017, which is a continuation-in-part of U.S. patent application Ser. No. 14/732,646, filed Jun. 5, 2015, each of which is entirely incorporated herein by reference.

### TECHNICAL FIELD

Various embodiments relate generally to home automation devices, and human biological signal gathering and analysis.

### BACKGROUND

According to current scientific research into sleep, there are two major stages of sleep: rapid eye movement (“REM”) sleep, and non-REM sleep. First comes non-REM sleep, followed by a shorter period of REM sleep, and then the cycle starts over again.

There are three stages of non-REM sleep. Each stage can last from 5 to 15 minutes. A person goes through all three stages before reaching REM sleep.

In stage one, a person's eyes are closed, but the person is easily woken up. This stage may last for 5 to 10 minutes. This stage is considered light sleep.

In stage two, a person is in light sleep. A person's heart rate slows and the person's body temperature drops. The person's body is getting ready for deep sleep. This stage is also considered light sleep.

Stage three is the deep sleep stage. A person is harder to rouse during this stage, and if the person was woken up, the person would feel disoriented for a few minutes. During the deep stages of non-REM sleep, the body repairs and regrows tissues, builds bone and muscle, and strengthens the immune system.

REM sleep happens 90 minutes after a person falls asleep. Dreams typically happen during REM sleep. The first period of REM typically lasts 10 minutes. Each of later REM stages gets longer, and the final one may last up to an hour. A person's heart rate and breathing quickens. A person can have intense dreams during REM sleep, since the brain is more active. REM sleep affects learning of certain mental skills.

Even in today's technological age, supporting healthy sleep is relegated to the technology of the past such as an electric blanket, a heated pad, or a bed warmer. The most advanced of these technologies, an electric blanket, is a blanket with an integrated electrical heating device which can be placed above the top bed sheet or below the bottom bed sheet. The electric blanket may be used to pre-heat the bed before use or to keep the occupant warm while in bed. However, turning on the electric blanket requires the user to remember to manually turn on the blanket, and then manually turn it on. Further, the electric blanket provides no additional functionality besides warming the bed.

### SUMMARY

Introduced are methods and systems for: gathering human biological signals, such as heart rate, breathing rate, or temperature; analyzing the gathered human biological signals; and controlling home appliances or a bed device system based on the analysis.

In one embodiment of the invention, one or more user sensors, associated with a piece of furniture, such as a bed, measure the bio signals associated with a user, such as the heart rate associated with said user or breathing rate associated with said user. One or more environment sensors measure the environment property such as temperature, humidity, light, or sound. Based on the bio signals associated with said user and environment properties received, the system determines the time at which to send an instruction to an alarm or an appliance to turn on or to turn off. In one embodiment, the appliance is a bed device, capable of heating or cooling the user's bed. In another embodiment, the appliance is a thermostat, a light, a coffee machine, or a humidifier.

In another embodiment of the invention, based on the heart rate, temperature, and breathing rate, associated with a user, the system determines the sleep phase associated with said user. Based on the sleep phase and the user-specified wake-up time, the system determines a time to wake up the user, so that the user does not feel tired or disoriented when woken up.

### BRIEF DESCRIPTION OF THE DRAWINGS

These and other objects, features and characteristics of the present embodiments will become more apparent to those skilled in the art from a study of the following detailed description in conjunction with the appended claims and drawings, all of which form a part of this specification. While the accompanying drawings include illustrations of various embodiments, the drawings are not intended to limit the claimed subject matter.

FIGS. 1A and 1B are diagrams of a bed device, according to embodiments.

FIGS. 2A and 2B illustrate examples of a bed device, according to embodiments.

FIG. 3 illustrates an example of layers comprising a bed device, according to one embodiment.

FIG. 4A illustrates a user sensor placed on a sensor strip, according to one embodiment.

FIG. 4B illustrates a user sensor placed on a sensor strip according to another embodiment.

FIG. 4C is a flowchart of a process to manufacture the body of the sensor strip, according to one embodiment.

FIG. 4D is a flowchart of a process to manufacture the tail part of the sensor strip, according to one embodiment.

FIGS. 5A, 5B, 5C, and 5D show different configurations of a sensor strip, to fit different size mattresses, according to one embodiment.

FIG. 6A illustrates the division of the heating coil into zones and subzones, according to one embodiment.

FIGS. 6B and 6C illustrate the independent control of the different subzones, according to one embodiment.

FIGS. 7A, 7B, and 7C are flowcharts of the process for deciding when to heat or cool the bed device, according to one embodiment.

FIG. 8 is a flowchart of the process for recommending a bed time to a user, according to one embodiment.

FIG. 9 is a flowchart of the process for activating the user's alarm, according to one embodiment.

FIGS. 10A and 10B are flowcharts of the process for turning off an appliance, according to embodiments.

FIG. 11 is a diagram of a system capable of automating the control of the home appliances, according to one embodiment.

FIG. 12 is an illustration of the system capable of controlling an appliance and a home, according to one embodiment.

FIG. 13 is a flowchart of the process for controlling an appliance, according to one embodiment.

FIG. 14 is a flowchart of the process for controlling an appliance, according to another embodiment.

FIG. 15 is a diagram of a system for monitoring biological signals associated with a user, and providing notifications or alarms, according to one embodiment.

FIG. 16 is a flowchart of a process for generating a notification based on a history of biological signals associated with a user, according to one embodiment.

FIG. 17 is a flowchart of a process for generating a comparison between a biological signal associated with a user and a target biological signal, according to one embodiment.

FIG. 18 is a flowchart of a process for detecting the onset of a disease, according to one embodiment.

FIG. 19 is a diagrammatic representation of a machine in the example form of a computer system within which a set of instructions, for causing the machine to perform any one or more of the methodologies or modules discussed herein, may be executed.

### DETAILED DESCRIPTION

Examples of a method, apparatus, and computer program for automating the control of home appliances and improving the sleep environment are disclosed below. In the following description, for the purposes of explanation, numerous specific details are set forth in order to provide a thorough understanding of the embodiments of the invention. One skilled in the art will recognize that the embodiments of the invention may be practiced without these specific details or with an equivalent arrangement. In other instances, well-known structures and devices are shown in block diagram form in order to avoid unnecessarily obscuring the embodiments of the invention.

### Terminology

Brief definitions of terms, abbreviations, and phrases used throughout this application are given below.

In this specification, the term “biological signal” and “bio signal” are synonyms, and are used interchangeably.

Reference in this specification to “sleep phase” means light sleep, deep sleep, or REM sleep. Light sleep comprises stage one and stage two, non-REM sleep.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described that may be exhibited by some embodiments and not by others. Similarly, various requirements are described that may be requirements for some embodiments but not others.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements. The coupling or connection between the elements can be physical, logical, or a combination thereof. For example, two devices may be coupled directly, or via one or more intermediary channels or devices. As another example, devices may be coupled in such a way that information can be passed there between, while not sharing any physical connection with one another. Additionally, the words “herein,” “above,” “below,” and words of similar import, when used in this application, shall refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list.

If the specification states a component or feature “may,” “can,” “could,” or “might” be included or have a characteristic, that particular component or feature is not required to be included or have the characteristic.

The term “module” refers broadly to software, hardware, or firmware components (or any combination thereof). Modules are typically functional components that can generate useful data or another output using specified input(s). A module may or may not be self-contained. An application program (also called an “application”) may include one or more modules, or a module may include one or more application programs.

The term “on top of” means that the two objects, where the first object is “on top of” the second object, can be rotated so that the first object is above the second object relative to the ground. The 2 objects can be in direct or indirect contact, or may not be in contact at all.

The terminology used in the Detailed Description is intended to be interpreted in its broadest reasonable manner, even though it is being used in conjunction with certain examples. The terms used in this specification generally have their ordinary meanings in the art, within the context of the disclosure, and in the specific context where each term is used. For convenience, certain terms may be highlighted, for example using capitalization, italics, and/or quotation marks. The use of highlighting has no influence on the scope and meaning of a term; the scope and meaning of a term is the same, in the same context, whether or not it is highlighted. It will be appreciated that the same element can be described in more than one way.

Consequently, alternative language and synonyms may be used for any one or more of the terms discussed herein, but special significance is not to be placed upon whether or not a term is elaborated or discussed herein. A recital of one or more synonyms does not exclude the use of other synonyms. The use of examples anywhere in this specification, including examples of any terms discussed herein, is illustrative only and is not intended to further limit the scope and meaning of the disclosure or of any exemplified term. Likewise, the disclosure is not limited to various embodiments given in this specification.

### Bed Device

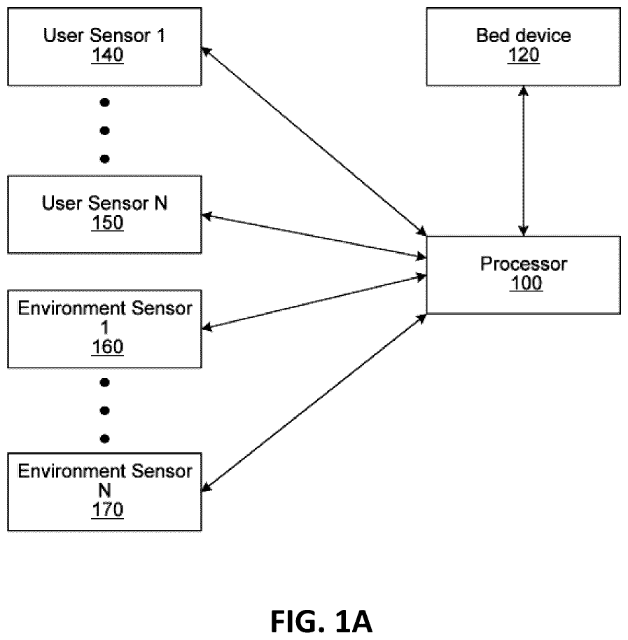

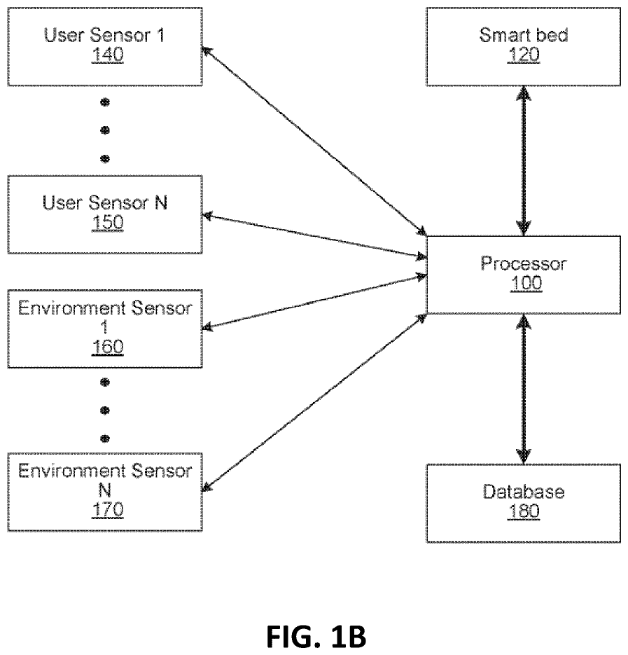

FIGS. 1A and 1B are of a diagram of a bed device, according to embodiments. Any number of user sensors 140, 150 monitor the bio signals associated with a user, such as the heart rate, the breathing rate, the temperature, motion, or presence, associated with said user. Any number of environment sensors 160, 170 monitor environment properties, such as temperature, sound, light, or humidity. The user sensors 140, 150 and the environment sensors 160, 170 communicate their measurements to the processor 100. The environment sensors 160, 170, measure the properties of the environment that the environment sensors 160, 170 are associated with. In one embodiment, the environment sensors 160, 170 are placed next to the bed. The processor 100 determines, based on the bio signals associated with said user, historical bio signals associated with said user, user-specified preferences, exercise data associated with said user, or the environment properties received, a control signal, and a time to send said control signal to a bed device 120.

According to one embodiment, the processor 100 is connected to a database 180, which stores the biological signals associated with a user. Additionally, the database 180 can store average biological signals associated with the user, history of biological signals associated with a user, etc. The database 180 can be associated with a user, or the database 180 can be associated with the bed device.

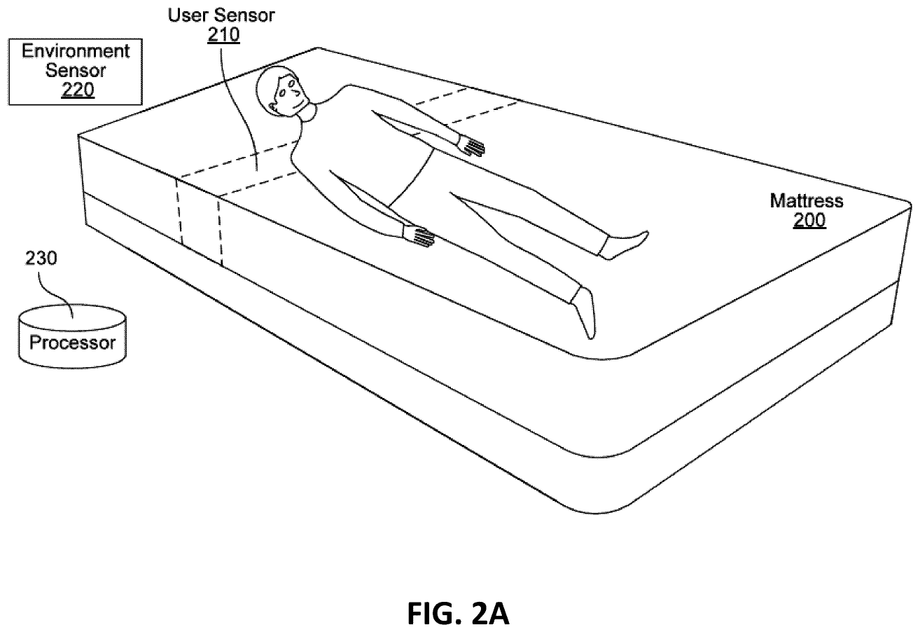

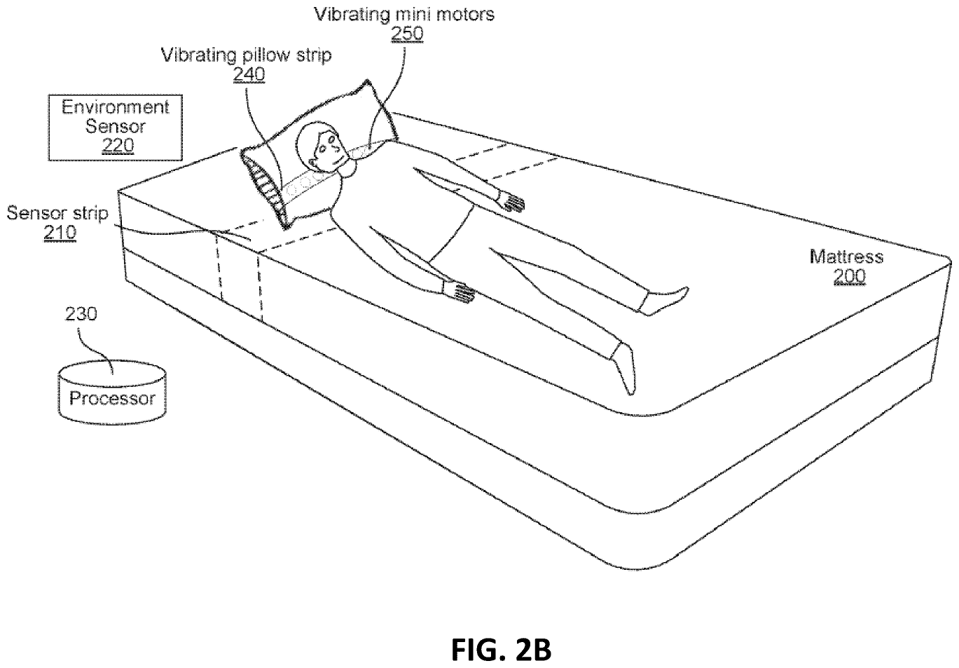

FIGS. 2A and 2B illustrate examples of the bed device of FIGS. 1A and 1B, according to embodiments. A user sensor 210, associated with a mattress 200 of the bed device 120, monitors bio signals associated with a user sleeping on the mattress 200. The user sensor 210 can be built into the mattress 200, or can be part of a bed device. Alternatively, the user sensor 210 can be a part of any other piece of furniture, such as a rocking chair, a couch, an armchair, etc. Based on the temperature of the user, user's presence in bed can be determined with substantially 100% accuracy. For example, when the temperature measured by the temperature sensor is within 35.5° C. to 37.5° C. range, a processor 230 can determine solely based on the temperature measurement, with substantially 100% accuracy, that the user is present in bed. The user sensor 210 comprises a temperature sensor, or a piezo sensor. The environment sensor 220 measures environment properties such as temperature, sound, light or humidity. According to one embodiment, the environment sensor 220 is associated with the environment surrounding the mattress 200. The user sensor 210 and the environment sensor 220 communicate the measured environment properties to the processor 230. In some embodiments, the processor 230 can be similar to the processor 100 of FIGS. 1A or 1B. A processor 230 can be connected to the user sensor 210, or the environment sensor 220 by a computer bus, such as an I2C bus. Also, the processor 230 can be connected to the user sensor 210, or the environment sensor 220 by a communication network. By way of example, the communication network connecting the processor 230 to the user sensor 210, or the environment sensor 220 includes one or more networks such as a data network, a wireless network, a telephony network, or any combination thereof. The data network may be any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, or any combination thereof. In addition, the wireless network may be, for example, a cellular network and may employ various technologies including enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., worldwide interoperability for microwave access (WiMAX), Long Term Evolution (LTE) networks, code division multiple access (CDMA), wideband code division multiple access (WCDMA), wireless fidelity (WiFi), wireless LAN (WLAN), Bluetooth®, Internet Protocol (IP) data casting, satellite, mobile ad-hoc network (MANET), and the like, or any combination thereof.

According to another embodiment, a vibrating pillow strip 240 is coupled to the user's pillow. The vibrating pillow strip 240 can be coupled to the pillow in various ways such as attached to the pillow case, attached to the surface of the pillow, attached to the filling inside the pillow, etc. The vibrating pillow strip 240 comprises a plurality of mini motors 250. The vibrating pillow strip 240 can also be attached to the mattress, a mattress pad, the sheets, any other piece of furniture, etc. According to one embodiment, a vibrating mini motor is a disk, at most 10 millimeters in diameter, and 2.7 mm in thickness. The vibrating mini motor is at most 0.9 g, and takes a voltage in the range of 2 and 5 V. At 5 V the mini motor vibrates at least at 11,000 rpm.

The processor 230 can be connected to the vibrating pillow strip 240 via a communication network. The data network may be any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, or any combination thereof. In addition, the wireless network may be, for example, a cellular network and may employ various technologies including enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., worldwide interoperability for microwave access (WiMAX), Long Term Evolution (LTE) networks, code division multiple access (CDMA), wideband code division multiple access (WCDMA), wireless fidelity (WiFi), wireless LAN (WLAN), Bluetooth®, Bluetooth low energy (BLE), Internet Protocol (IP) data casting, satellite, mobile ad-hoc network (MANET), and the like, or any combination thereof.

The processor 230 is any type of microcontroller, or any processor in a mobile terminal, fixed terminal, or portable terminal including a mobile handset, station, unit, device, multimedia computer, multimedia tablet, Internet node, cloud computer, communicator, desktop computer, laptop computer, notebook computer, netbook computer, tablet computer, personal communication system (PCS) device, personal navigation device, personal digital assistants (PDAs), audio/video player, digital camera/camcorder, positioning device, television receiver, radio broadcast receiver, electronic book device, game device, the accessories and peripherals of these devices, or any combination thereof.

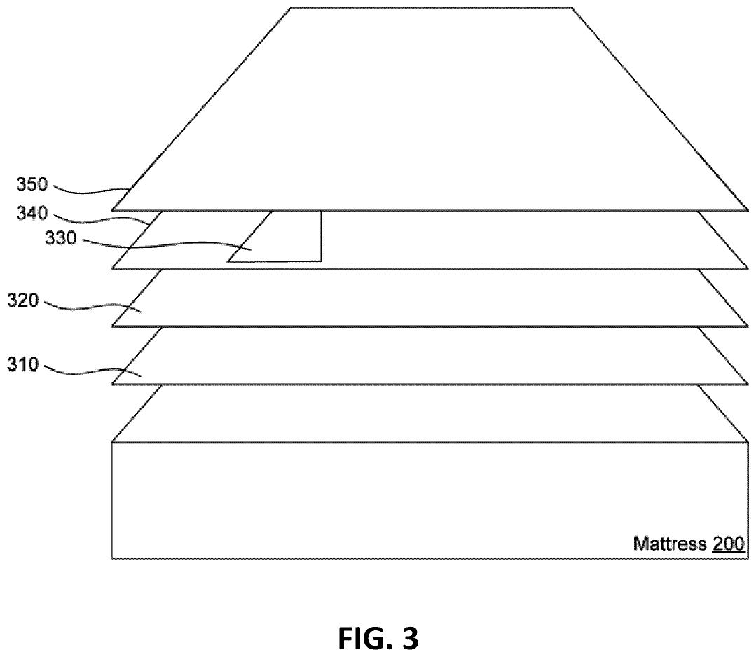

FIG. 3 illustrates an example of layers comprising the bed device (e.g., a bed pad device) of FIGS. 1A and 1B, according to one embodiment. In some embodiments, the bed device 120 is a pad that can be placed on top of the mattress 200. Bed device 120 comprises a number of layers. A top layer 350 comprises fabric. A layer 340 comprises batting, and a sensor strip 330. A layer 320 comprises coils for cooling or heating the bed device. A layer 310 comprises waterproof material.

According to another embodiment, the layer 320 comprises a material that can be heated or cooled in the 10° C. to 50° C. range without changing the materials properties such as the state of matter. An example of such materials can be air, water, argon, a synthetic material such as carbon nanotubes, etc. According to one embodiment, the layer 320 is connected to an external thermal regulator which heats or cools the material, based on the signal received from the processor 230.

According to another embodiment, the layer 320 comprising the material is integrated into the mattress, the bed sheets, the bed cover, the bed frame, etc. The layer 320 comprising the material can also be integrated with any piece of furniture.

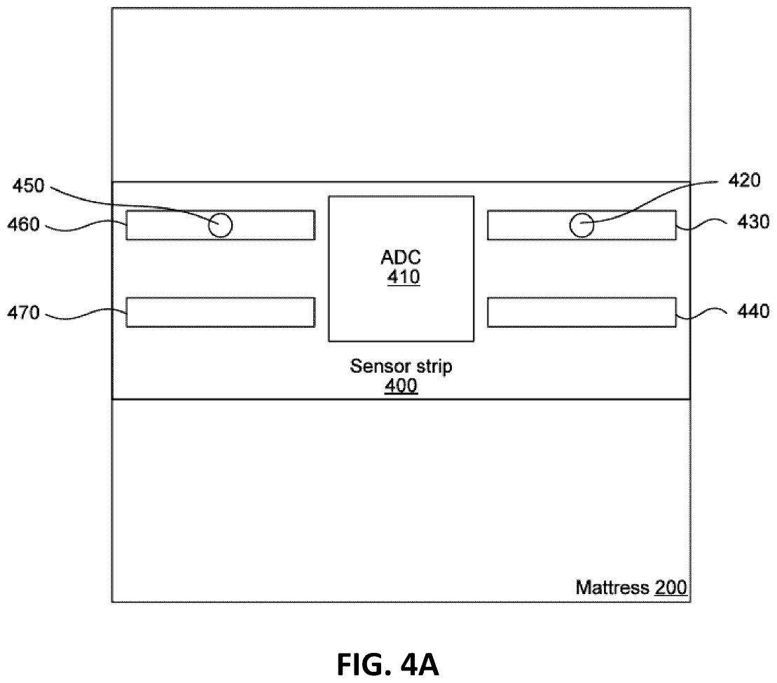

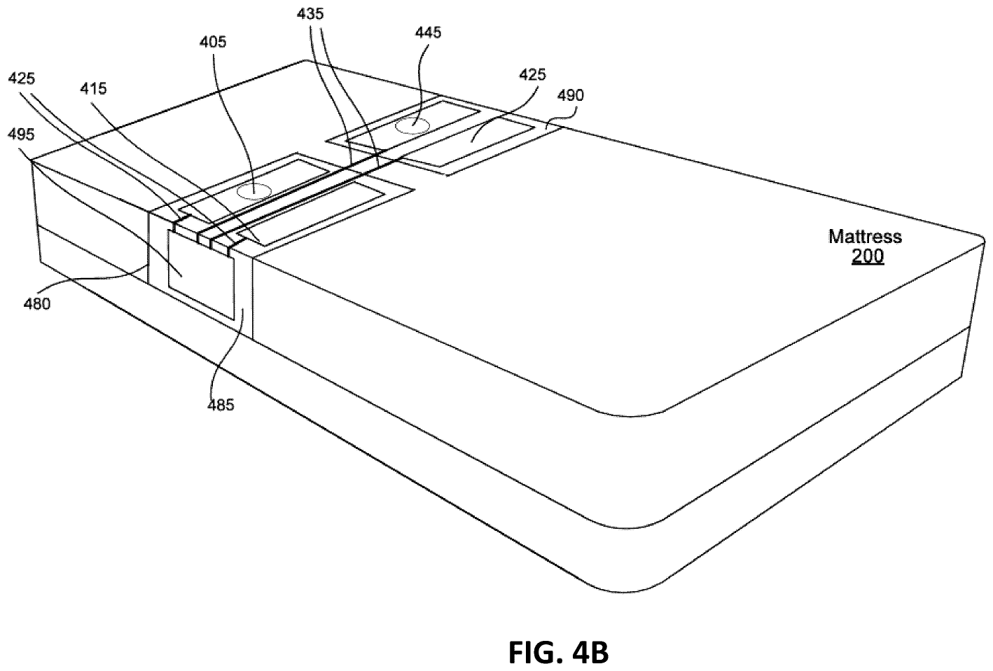

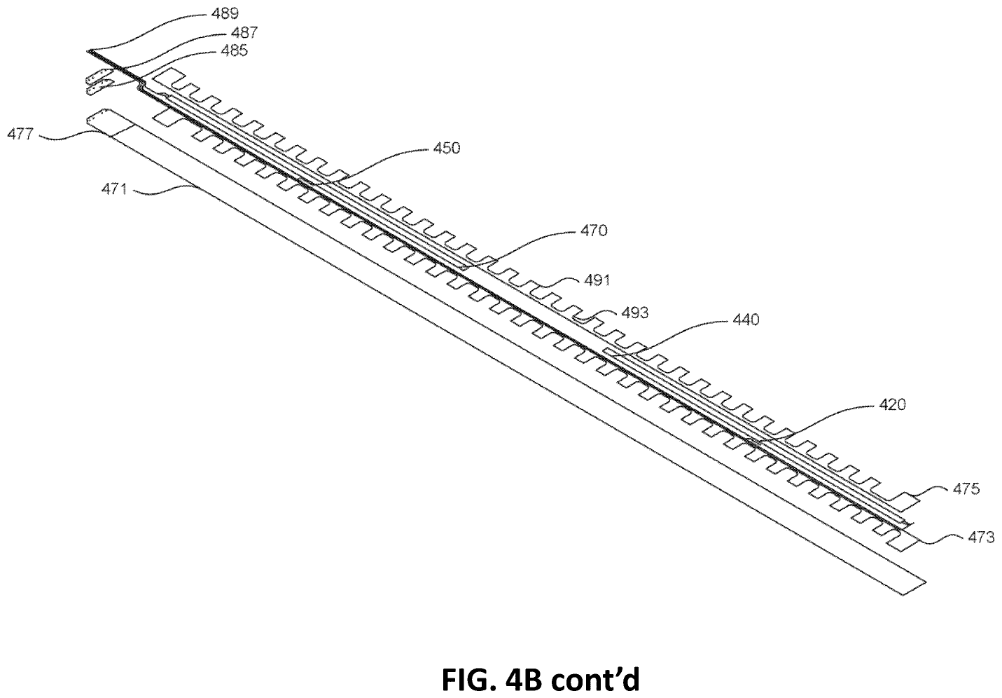

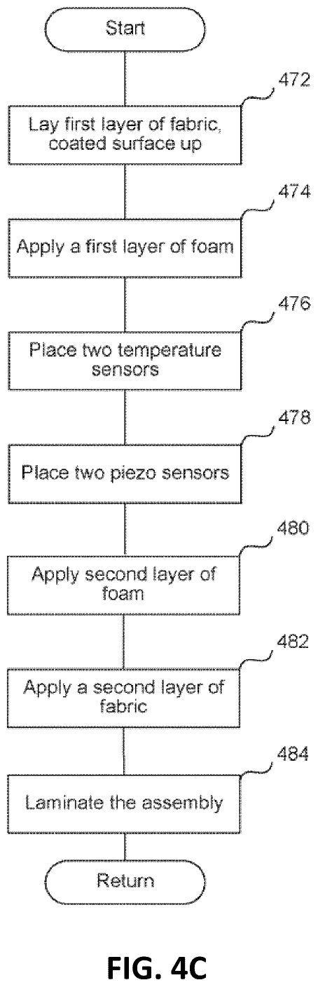

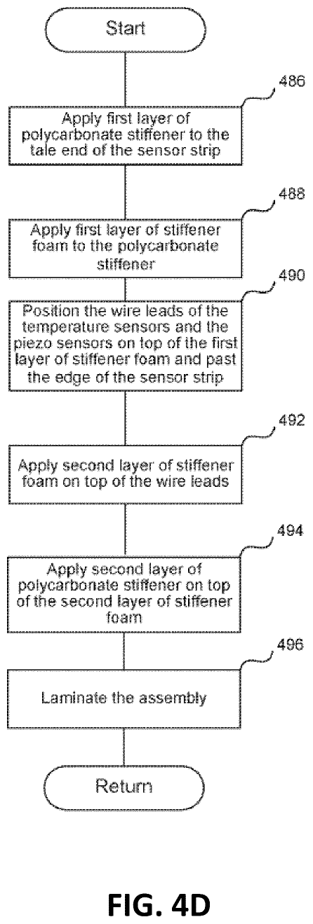

FIG. 4A illustrates a user sensor 420, 440, 450, 470 placed on a sensor strip 400, according to one embodiment. In some embodiments, the user sensors 420, 440, 450, 470 can be similar to or part of the user sensor 210 of FIGS. 2A and 2B. Sensors 470 and 440 comprise a piezo sensor, which can measure a bio signal associated with a user, such as the heart rate and the breathing rate. Sensors 450 and 420 comprise a temperature sensor. According to one embodiment, sensors 450, and 470 measure the bio signals associated with one user, while sensors 420, 440 measure the bio signals associated with another user. Analog-to-digital converter 410 converts the analog sensor signals into digital signals to be communicated to a processor. Computer bus 430 and 460, such as the I2C bus, communicates the digitized bio signals to a processor.

FIG. 4B illustrates a user sensor placed on a sensor strip according to another embodiment. The sensor strip 480 includes two sections 485, 490. Each sensor strip section 485, 490 includes a temperature sensor 405, 445, respectively, and a piezo sensor 415, 425, respectively. The temperature sensors 405, 445 and the piezo sensors 415, 425 are connected to the analog-to-digital converter 495 using wires 425, 435 respectively. The analog-to-digital converter 495 is placed on the side of the strip. In other embodiments, there can be multiple analog-to-digital converters placed on the strip, where the multiple analog-to-digital converters correspond to each sensor strip section 485, 490. In various embodiments, there can be a plurality of sensors strips 480, 400 associated with the mattress 200. Each sensor strip section 485, 490 includes multiple protrusions 491, and multiple gaps 493, between the multiple protrusions 491. The sensor strip 400 comprises several layers, such as a fabric layer 471, a foam layer 473, 475, a piezo sensor 470, 440, a polycarbonate stiffener 485, a stiffener foam 487, and a temperature sensor 450, 420. Region 477 of the fabric layer 471 is the tail region of the sensor strip 400. Wire leads 489 associated with piezo sensor 470, 440, and temperature sensor 450, 420 are placed on top of the tail region 477. The fabric layer includes two short edges and two long edges. The length of the short edge varies from 40-70 mm. The fabric layer 471 has at least one coated surface. The foam layer 473, 475 also has two short edges and two long edges. One of the long edges includes multiple protrusions 491, and multiple gaps 493, between the multiple protrusions 491. FIG. 4C is a flowchart of a process to manufacture the body of the sensor strip 400, according to one embodiment. In step 472, the fabric layer 471 is laid out with the coated surface pointing up. In step 474, a first foam layer is applied to the fabric layer 471. In one embodiment, the first foam layer 473 is centered on the fabric layer 471, with a margin of 10 mm from the first short edge and a margin of 5 mm from the long edges. The margin to the second short edge of the fabric layer 471 is greater than the margin to the first short edge. In one embodiment, the margin to the second short edge is at least twice as big than the margin to the first short edge. The margin to the second short edge of the fabric layer 471 is considered a tail part of the sensor strip 400, comprising the tail region 477 of the fabric layer 471. In step 476, two temperature sensors 450, 420 are placed on the first foam layer 473. In one embodiment, the temperature sensors are placed 17 mm from a long edge of the fabric layer 471. In step 478, two piezo sensors 470, 440 are placed on the first foam layer 473. In one embodiment, the piezo sensors are centered on the fabric layer 471. In step 480, a second foam layer 475 is applied on top of the piezo sensors. In one embodiment, the second foam layer 475 is centered on the fabric layer 471, with a margin of 10 mm from the short edges, and 5 mm from the long edges. Further, the second foam layer 475 is placed as a mirror image of the first foam layer 473, and is interlaced with the first foam layer 473. In step 482, a second fabric layer is applied on top of the second foam layer 475. In step 484, the whole assembly, comprising all the layers, is laminated.

FIG. 4D is a flowchart of a process to manufacture the tail part of the sensor strip 400, according to one embodiment. In step 486, first polycarbonate stiffener layer 485 is placed on top of the tail region 477 of the fabric layer 471. In one embodiment, the dimensions of the polycarbonate stiffener layer 485 are 40-70 mm by 5-25 mm. The 40-70 mm edge matches the length of the 40-70 mm edge of the sensor strip 400. In step 488, the first stiffener foam layer 487 is applied on top of the polycarbonate stiffener layer 485. In step 490, the wire leads 489 of the piezo sensors 470, 440, and the wire leads 489 of the temperature sensors 450, 420 are placed on top of the first stiffener foam layer 487, and past the tail region 477 of the fabric layer 471. In step 492, the second stiffener foam layer is applied on top of the wire leads 489. The dimensions of the second stiffener foam layer are identical to the first stiffener foam layer 487. In step 494, the second polycarbonate stiffener layer is applied on top of the second stiffener foam layer. The dimensions of the second polycarbonate stiffener layer are identical to the dimensions of the first polycarbonate stiffener layer 485. In step 496, the whole tail part assembly is laminated.

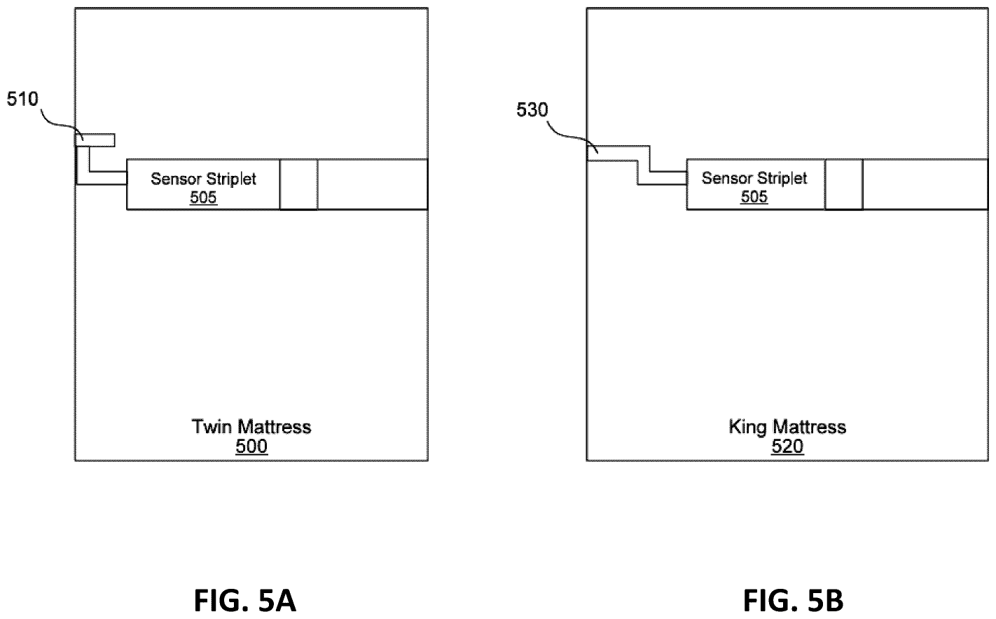

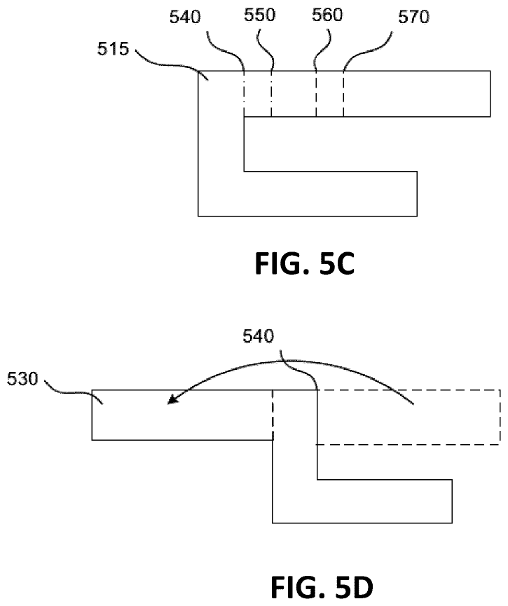

FIGS. 5A and 5B show different configurations of the sensor strip, to fit different size mattresses, according to one embodiment. FIGS. 5C and 5D show how such different configurations of the sensor strip can be achieved. Specifically, sensor strip 400 comprises a computer bus 510, 530, and a sensor striplet 505. The computer bus 510, 530 can be bent at predetermined locations 540, 550, 560, 570. Bending the computer bus 515 at location 540 produces the maximum total length of the computer bus 530. Computer bus 530 combined with a sensor striplet 505, fits a king size mattress 520. Bending the computer bus 515 at location 570 produces the smallest total length of the computer bus 510. Computer bus 510 combined with a sensor striplet 505, fits a twin size mattress 500. Bending the computer bus 515 at location 560, enables the sensor strip 400 to fit a full-size bed. Bending the computer bus 515 at location 550 enables the sensor strip 400 to fit a queen-size bed. In some embodiments, twin mattress 500, or king mattress 520 can be similar to the mattress 200 of FIGS. 2A and 2B.

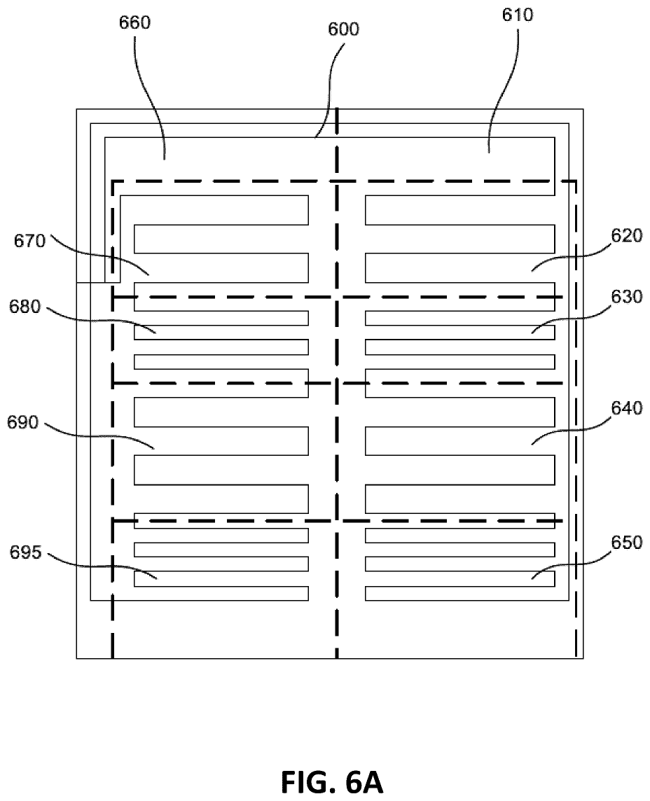

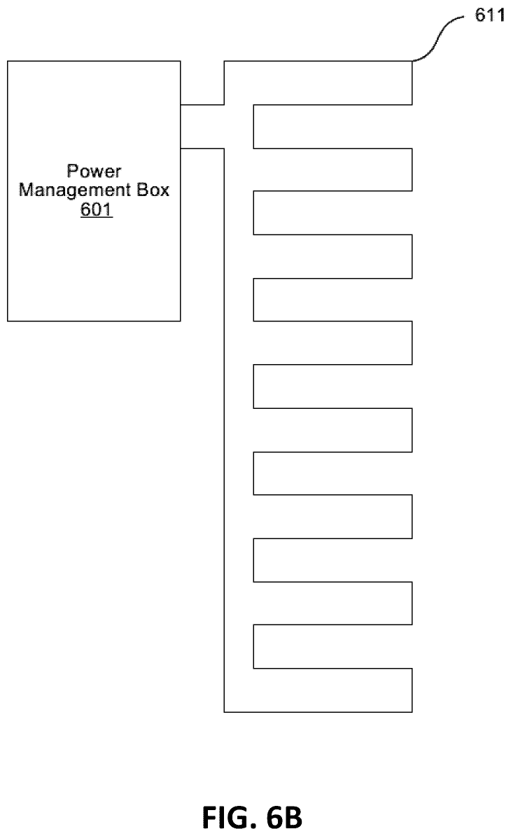

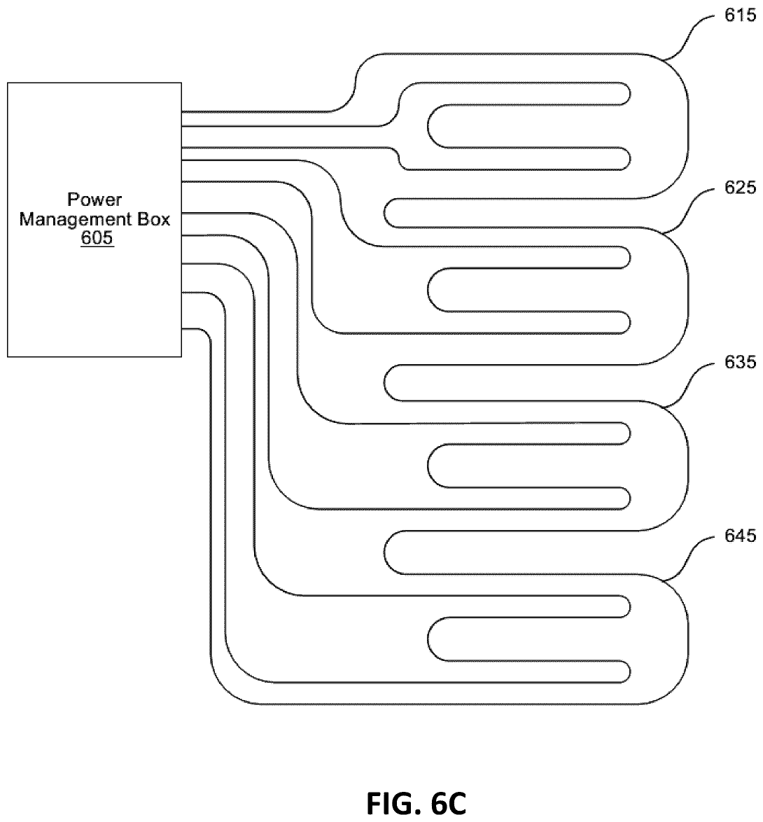

FIG. 6A illustrates the division of the heating coil 600 into zones and subzones, according to one embodiment. Specifically, the heating coil 600 is divided into two zones 660 and 610, each corresponding to one user of the bed. Each zone 660 and 610 can be heated or cooled independently of the other zone in response to the user's needs. To achieve independent heating of the two zones 660 and 610, the power supply associated with the heating coil 600 is divided into two zones, each power supply zone corresponding to a single user zone 660, 610. Further, each zone 660 and 610 is further subdivided into subzones. Zone 660 is divided into subzones 670, 680, 690, and 695. Zone 610 is divided into subzones 620, 630, 640, and 650. The distribution of coils in each subzone is configured so that the subzone is uniformly heated. However, the subzones may differ among themselves in the density of coils. For example, the data associated with said user subzone 670 has lower density of coils than subzone 680. This will result in subzone 670 having lower temperature than subzone 680, when the coils are heated. Similarly, when the coils are used for cooling, subzone 670 will have higher temperature than subzone 680. According to one embodiment, subzones 680 and 630 with highest coil density correspond to the users' lower backs; and subzones 695 and 650 with highest coil density correspond to the users' feet.

According to one embodiment, even if the users switch sides of the bed, the system will correctly identify which user is sleeping in which zone by identifying the user based on any of the following signals alone, or in combination: heart rate, breathing rate, body motion, or body temperature associated with said user. The system can also identify the user by receiving from a user device associated with the user an identification (ID) associated with the user. For example, the user can specify the user ID of the person sleeping on the sensor strip. If there are multiple sensor strips and/or multiple sensors, the user can specify the ID of the person associated with each sensor strip and/or each sensor.

In another embodiment, the power supply associated with the heating coil 600 is divided into a plurality of zones, each power supply zone corresponding to a subzone 620, 630, 640, 650, 670, 680, 690, 695. The user can control the temperature of each subzone 620, 630, 640, 650, 670, 680, 690, 695 independently. Further, each user can independently specify the temperature preferences for each of the subzones. Even if the users switch sides of the bed, the system will correctly identify the user, retrieve the user identification (user ID), and the preferences associated with the user by identifying the user based on any of the following signals alone, or in combination: heart rate, breathing rate, body motion, or body temperature associated with said user. According to another embodiment, if the users switch sides of the bed, the system receives the user ID of the new user from a user device associated with the user, and retrieves the preferences associated with the user.

FIGS. 6B and 6C illustrate the independent control of the different subzones in each zone 610, 660, according to one embodiment. A set of uniform coils 611, connected to power management box 601, uniformly heats or cools the bed. Another set of coils, targeting specific areas of the body such as the neck, the back, the legs, or the feet, is layered on top of the uniform coils 611. Subzone 615 heats or cools the neck. Subzone 625 heats or cools the back. Subzone 635 heats or cools the legs, and subzone 645 heats or cools the feet. Power is distributed to the coils via duty cycling of the power supply 605. Contiguous sets of coils can be heated or cooled at different levels by assigning the power supply duty cycle to each set of coils. The user can control the temperature of each subzone independently.

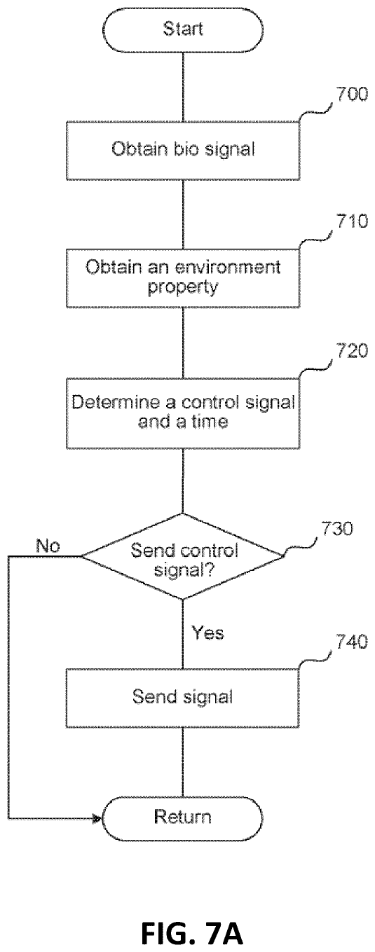

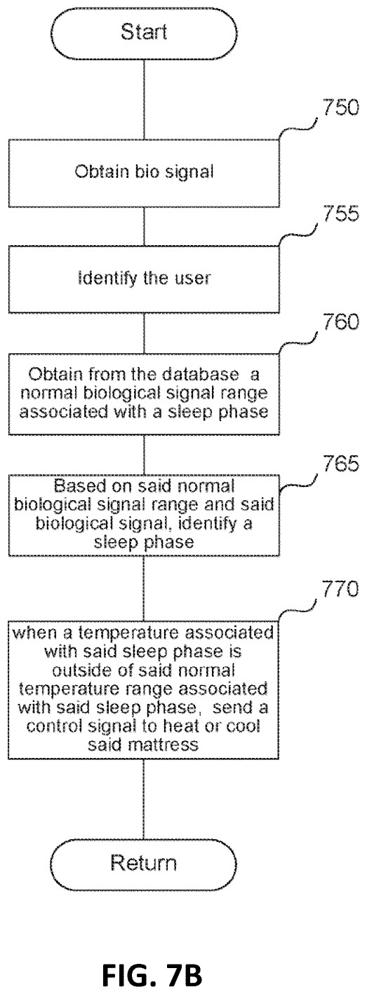

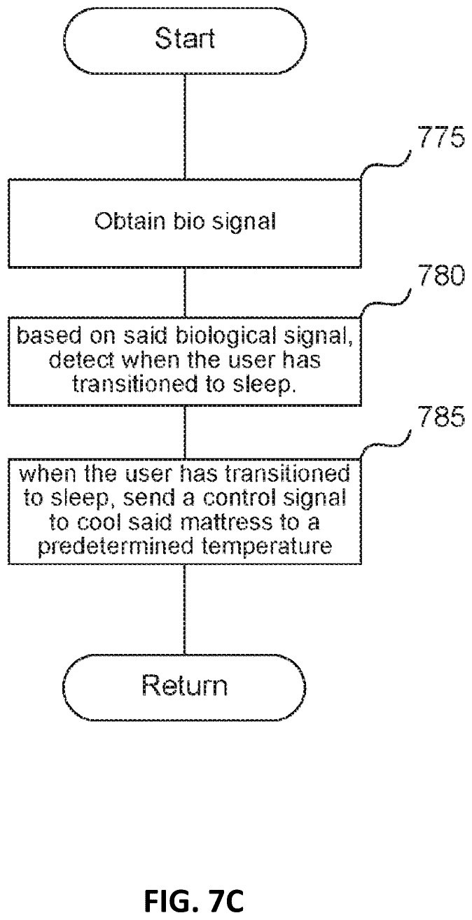

FIGS. 7A-7C are flowcharts of the process for deciding when to heat or cool the bed device, according to embodiments. At block 700, the process obtains a biological signal associated with a user, such as presence in the bed, motion, breathing rate, heart rate, or a temperature. The process obtains said biological signal from a sensor associated with a user. Further, at block 710, the process obtains the environment property, such as the amount of ambient light and the bed temperature. The process obtains the environment property from the environment sensor associated with the bed device.

At block 720, the process determines the control signal and the time to send a control signal. At block 730, the process sends the control signal to the bed device. For example, if the user is in bed, the bed temperature is low, and the ambient light is low, the process sends a control signal to the bed device. The control signal comprises an instruction to heat the bed device to the average nightly temperature associated with the user. According to another embodiment, the control signal comprises an instruction to heat the bed device to a user-specified temperature. Similarly, if the user is in bed, the bed temperature is high, and the ambient light is low, the process sends a control signal to the bed device to cool the bed device to the average nightly temperature associated with the user. According to another embodiment, the control signal comprises an instruction to cool the bed device to a user-specified temperature.

In another embodiment, in addition to obtaining the biological signal associated with the user, and the environment property, the process obtains a history of biological signals associated with the user. The history of biological signals can be stored in a database associated with the bed device, or in a database associated with a user. The history of biological signals comprises the average bedtime the user went to sleep for each day of the week; that is, the history of biological signals comprises the average bedtime associated with the user on Monday, the average bedtime associated with the user on Tuesday, etc. For a given day of the week, the process determines the average bedtime associated with the user for that day of the week, and sends the control signal to the bed device, allowing enough time for the bed to reach the desired temperature, before the average bedtime associated with the user. The control signal comprises an instruction to heat, or cool the bed to a desired temperature. The desired temperature may be automatically determined, such as by averaging the historical nightly temperature associated with a user, or the desired temperature may be specified by the user.

FIG. 7B is a flowchart of the process for cooling or heating a bed device, according to another embodiment. In step 750, processor 230 obtains the biological signal associated with the user, wherein the biological signal comprises a respiration rate associated with the user, a heart rate associated with the user, a motion associated with the user, or a temperature associated with the user. In step 755, the processor 230 identifies the user based on at least one of: the heart rate associated with the user, the respiration rate associated with the user, the motion associated with the user, or the temperature associated with the user. In step 760, based on the user identification, the processor 230 obtains from the database 180 a normal biological signal range associated with a sleep phase in a plurality of sleep phases associated with the user, wherein the normal biological signal range comprises a normal temperature range associated with the user. In step 765, based on the normal biological signal range and the biological signal, the processor 230 identifies a sleep phase in a plurality of sleep phases associated with the user. The plurality of sleep phases includes the sleep phase comprising a wakefulness phase, a light sleep phase, a deep sleep phase, or a rapid eye movement sleep phase. In step 770, when the temperature associated with the sleep phase is outside of the normal temperature range associated with the sleep phase, the processor 230 sends a control signal to a temperature control device coupled to the mattress, the control signal comprising an instruction to heat or cool the mattress to a temperature within the normal temperature range.

According to one embodiment, the processor 230 obtains the biological signal associated with a user from the sensor strip 210 coupled to the mattress, where the sensor strip 210 measures the biological signal associated with the user. In another embodiment, the processor 230 obtains the biological signal associated with the user from a wearable device coupled to the user, which measures the users biological signals, such as a fitbit bracelet. The processor 230 can also store the biological signals into the database 180.

According to another embodiment, the processor 230 determines a current time. The processor 230 identifies the user based on at least one of: the heart rate associated with the user, the respiration rate associated with the user, the motion associated with the user, or the temperature associated with the user. Based on the user identification, the processor 230 obtains a wake-up time associated with the user. When the current time is at most 3 hours before the wake-up time, the processor 230 sends the control signal to the temperature control device coupled to the mattress, the control signal comprising an instruction to turn off.

The processor 230 can detect a sleep phase by detecting a slowdown in the heart rate, a drop in the temperature, and a regular respiration rate. The processor 230 can also detect the sleep phase by detecting an end to preceding sleep phase. For example, a healthy user normally cycles between light sleep, deep sleep and REM sleep, in sequence, throughout the night. When the REM sleep phase ends, the light sleep phase begins, followed by a deep sleep phase.

According to another embodiment, the processor 230 obtains perspiration associated with the user from a perspiration sensor built into the sensor strip 210. When the user is perspiring, the processor sends a control signal to cool the temperature control device by a fraction of a degree Celsius, until the user stops perspiring. The processor 230 maintains the temperature at which the user is not perspiring. The fraction of a degree Celsius can be 1/10, ⅕, ¼, ½, 1, etc. According to another embodiment, based on the total amount of perspiration from the user during the sleep, the processor 230 recommends an amount of liquid, such as water or electrolytes, that the user should consume upon waking up.

According to another embodiment, the processor 230 sends a control signal to cool or heat the temperature control device of a fraction of a degree Celsius, and monitors the quality of users sleep. For example, the processor 230 monitors if the user goes through the sleep cycles in order, and if the sleep cycles last a normal amount of time. Once the user sleep cycles becomes irregular, or do not last a normal amount of time, the processor records the last temperature, at which the user slept soundly. The last temperature at which the user slept soundly is the limit of the comfortable temperature range associated with that user. The limit can be a high temperature limit, or a low temperature limit. The fraction of a degree Celsius can be 1/10, ⅕, ¼, ½, 1, etc. The processor 230 stores the comfortable temperature range associated with the user, comprising a high temperature limit, and a low temperature limit, and heats or cools the bed to temperature within the comfortable temperature range.

FIG. 7C is a flowchart of the process for cooling or heating a bed device, according to yet another embodiment. In step 775, the processor 230 obtains the biological signal associated with the user, wherein the biological signal comprises a respiration rate associated with the user, a heart rate associated with the user, a motion associated with the user, or a temperature associated with the user. In step 780, based on the biological signal, the processor 230 detects when the user has transitioned to sleep. The processor 230 detects transition to sleep by detecting a slowdown in the heart rate, a regular heart rate, a drop in the temperature, and/or a regular respiration rate. In step 785, when the user has transitioned to sleep, the processor 230 sends a control signal to a temperature control device coupled to the mattress, the control signal comprising an instruction to cool the mattress to a predetermined temperature. The predetermined temperature can be the average nightly temperature associated with the user, the predetermined temperature can be in the range 27 to 35° C., or the temperature can be user-specified. The biological signal can be measured by the sensor strip 210, or by any other sensing device, such as a wearable sensor, e.g. a fitbit bracelet.

According to another embodiment, the processor 230 obtains an ambient temperature surrounding the user. The environment sensor 220 can supply the processor 230 with the ambient temperature. When the ambient temperature is outside of a 35° C. to 36° C. range, the processor 230 sends the control signal to the temperature control device coupled to the mattress, the control signal comprising an instruction to adjust the mattress to a temperature within 27° C. to 35° C. range, or a user-specified temperature.

According to another embodiment, the processor 230 identifies the user based on at least one of: the heart rate associated with the user, the respiration rate associated with the user, the temperature associated with the user, or the motion associated with the user. Based on the user identification, the processor 230 determines an average bedtime associated with the user. The average bedtime can be the same for every day of the week, or can comprise an average Monday bedtime, an average Tuesday bedtime, an average Wednesday bedtime, an average Thursday bedtime, an average Friday bedtime, an average Saturday bedtime, or an average Sunday bedtime. At the average bedtime associated with the user, the processor 230 sends the control signal to the temperature control device coupled to the mattress, wherein the control signal comprises one of an instruction to heat the temperature control device to a temperature in a 27° C. to 35° C. range, or an instruction to cool the temperature control device to the temperature in the 37° C. to 35° C. range. The temperature can be a user-specified temperature.

### Bio Signal Processing

The technology disclosed here categorizes the sleep phase associated with a user as light sleep, deep sleep, or REM sleep. Light sleep comprises stage one and stage two sleep. The technology performs the categorization based on the breathing rate associated with said user, heart rate associated with said user, motion associated with said user, and body temperature associated with said user. Generally, when said user is awake, the breathing is erratic. When the user is sleeping, the breathing becomes regular. The transition between being awake and sleeping is quick and lasts less than 1 minute. The user cycles through light sleep, deep sleep, and REM sleep throughout the night. A complete sleep cycle takes on average between 90 and 110 minutes.

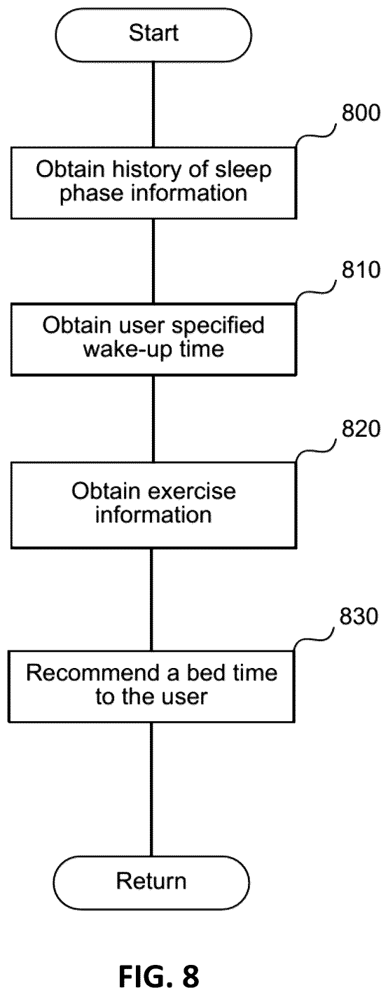

FIG. 8 is a flowchart of the process for recommending a bed time to the user, according to one embodiment. At block 800, the process obtains a history of sleep phase information associated with said user. The history of sleep phase information comprises an amount of time the user spent in each of the sleep phases, light sleep, deep sleep, or REM sleep. The history of sleep phase information can be stored in a database associated with the user. Based on this information, the process determines how much light sleep, deep sleep, and REM sleep the user needs on average every day. In another embodiment, the history of sleep phase information comprises the average bedtime associated with said user for each day of the week (e.g., the average bedtime associated with said user on Monday, the average bedtime associated with said user on Tuesday, etc.). At block 810, the process obtains user-specified wake-up time, such as the alarm setting associated with said user. At block 820, the process obtains exercise information associated with said user, such as the distance the user ran that day, the amount of time the user exercised in the gym, or the amount of calories the user burned that day. According to one embodiment, the process obtains said exercise information from a user phone, a wearable device, a fitbit bracelet, or a database storing said exercise information. Based on all this information, at block 830, the process recommends a bedtime to the user. For example, if the user has not been getting enough deep and REM sleep in the last few days, the process recommends an earlier bedtime to the user. Also, if the user has exercised more than the average daily exercise, the process recommends an earlier bedtime to the user.

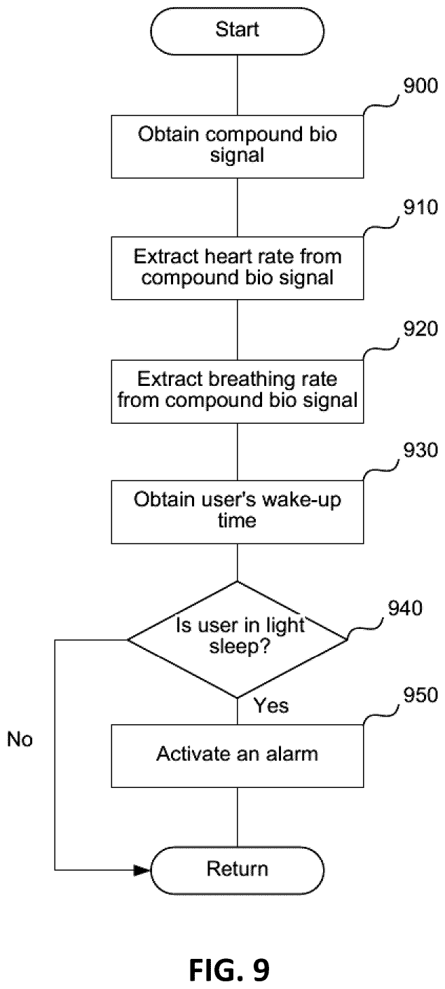

FIG. 9 is a flowchart of the process for activating a user's alarm, according to one embodiment. At block 900, the processor 230 obtains the compound bio signal associated with said user. The compound bio signal associated with said user comprises the heart rate associated with said user. The compound bio signal associated with the user comprises the heart rate associated with the user, the respiration rate associated with the user, the motion associated with the user, and the temperature associated with the user. According to one embodiment, the processor 230 obtains the compound bio signal from a sensor associated with said user. At block 910, the processor 230 extracts the heart rate signal from the compound bio signal. For example, the processor 230 extracts the heart rate signal associated with said user by performing low-pass filtering on the compound bio signal. Also, at block 920, the processor 230 extracts the respiration rate signal from the compound bio signal. For example, the processor 230 extracts the breathing rate by performing bandpass filtering on the compound bio signal. The respiration rate signal includes breath duration, pauses between breaths, as well as breaths per minute. The processor 230 also extracts the temperature signal and the motion signal from the compound bio signal. At block 930, the processor 230 obtains user's wake-up time, such as the alarm setting associated with said user. In order to obtain the user's wake-up time, the processor 230, first identifies the user based on the user's bio signal. Based on the heart rate signal and the breathing rate signal, the processor 230 determines the sleep phase associated with said user, and if the user is in light sleep, and current time is at most one hour before the alarm time, at block 940, the processor 230 sends a control signal to an alarm. Waking up the user during the deep sleep or REM sleep is detrimental to the user's health because the user will feel disoriented, groggy, and will suffer from impaired memory. Consequently, at block 950, the processor 230 activates an alarm, when the user is in light sleep and when the current time is at most one hour before the user-specified wake-up time.

According to another embodiment, the process obtains the user's wake-up time, such as the alarm setting associated with the user, and at the user's wake-up time sends a control signal to the alarm to activate.

The alarm can be a vibrating pillow strip 240 coupled to the user, for example a vibrating pillow strip 240 attached to the pillow, to the pillowcase, mattress, sheets, attached to a bed pad, attached to the user's cover, or any other piece of furniture etc. In one embodiment, the alarm can be a wearable device attached to the user, such as a bracelet. The vibrating pillow strip 240 can be divided into a plurality of zones corresponding to a plurality of users. For example, the left side of the bed corresponds to zone 1, and the right side of the bed corresponds to zone 2. Zone 1 and zone 2 can vibrate independently of each other. When the vibrating pillow strip 240 is divided into the plurality of zones, the control signal comprises an identification associated with the zone to which the control signal is sent.

According to one embodiment, the vibrating pillow strip 240 includes a plurality of vibrating mini motors 250, attached to the strip. The vibrating pillow strip 240 can receive a control signal instructing the vibrating mini motors 250 to vibrate. The vibrating mini motors 250 can be configured to vibrate synchronously, or they can be configured to vibrate asynchronously, for example vibrating in order from left to right. The vibrating mini motors 250 are designed to be small enough to be unnoticeable by the sleeping user, and to be powerful enough to wake up the sleeping user.

The process can detect whether the user is in light sleep in several ways. According to one embodiment, the process detects that user is in light sleep if within a period of at most 5 minutes there is a slow-down in the user's heart rate, a drop in the user's temperature, and the users respiration (or breathing as used interchangeably herein) becomes regular. According to another embodiment, the process detects that the user is in light sleep if the user is sleeping, and the rapid eye movement sleep phase has ended. In another embodiment, the database 180 stores a history of biological signals associated with the user, wherein the history of biological signals associated with the user comprises a normal heart rate range associated with each sleep phase, a normal respiration rate range associated with each sleep phase, a normal motion range associated with each sleep phase, and a normal temperature range associated with each sleep phase. The process obtains from the database 180 the history of biological signals associated with a user. To obtain the history of bio signals associated with a user, the process first identifies the user based on the current bio signal associated with the user. The current bio signal comprises the current respiration rate, the current temperature in the current motion associated with the user. Based on the history of bio signals in the current bio signal, the process determines the best match between the current bio signal and the history of bio signals associated with each sleep phases. If the best match between the current bio signal and the history of bio signals is light sleep, the process determines that the user is in light sleep. According to one embodiment, the best match is determined by least square difference between the current bio signal and the history of bio signals.

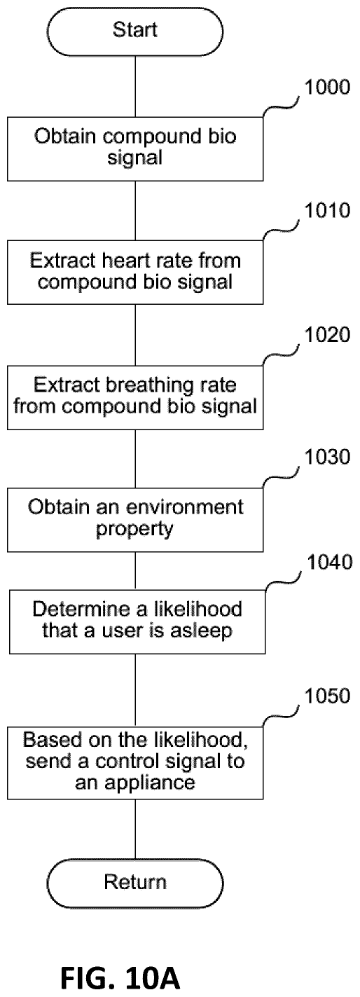

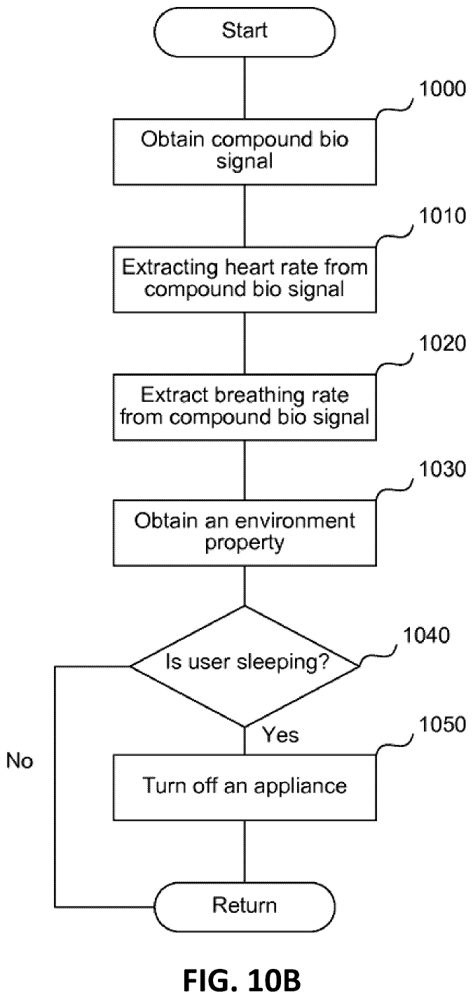

FIGS. 10A and 10B are flowcharts of the process for turning off an appliance, according to embodiments. At block 1000, the process obtains the compound bio signal associated with said user. The compound bio signal comprises the heart rate associated with said user, and the breathing rate associated with said user. According to one embodiment, the process obtains the compound bio signal from a sensor associated with said user. At block 1010, the process extracts the heart rate signal from the compound bio signal by, for example, performing low-pass filtering on the compound bio signal. Also, at block 1020, the process extracts the breathing rate signal from the compound bio signal by, for example, performing bandpass filtering on the compound bio signal. At block 1030, the process obtains an environment property, comprising an ambient temperature, a humidity, a light intensity, a sound intensity, and/or a current time from an environment sensor associated with said user sensor. Based on the environment property and the sleep state associated with said user, at block 1040, the process determines whether (e.g., the likelihood that) the user is sleeping. If the user is sleeping, the processor 230, at block 1050, turns an appliance off. For example, if the user is asleep and the environment temperature is above the average nightly temperature, the processor 230 turns off the thermostat. Further, if the user is asleep and the lights are on, the processor 230 turns off the lights. Similarly, if the user is asleep and the TV is on, the processor 230 turns off the TV.

In one embodiment, the process keeps track of a likelihood that the user is asleep. The process can take into account the environment property and/or the sleep state associated with the user to determine the likelihood that the user is asleep. If the likelihood that the user is asleep is above a specified threshold, such as above 0.5, the process determines that the user is asleep, and takes corresponding actions.

To calculate the likelihood that the user is asleep, the process takes into account the environment property such as the current time, and compares the current time to the average bedtime associated with the user. If the current time is greater than the average bedtime associated with the user, the process increases the likelihood that the user is asleep. If the light intensity is lower than an average light intensity associated with the space where the environment sensor is placed, the process also increases the likelihood that the user is asleep. If the sound intensity is lower than an average sound intensity associated with the space where the environment sensor is placed, the process increases the likelihood that the user is asleep. Similarly, if the light intensity is higher than the average light intensity, the process decreases the likelihood that the user is asleep, and if the sound intensity is higher than the average sound intensity, the process decreases the likelihood that the user is asleep. The average associated with the environment property, such as the average light intensity and the average sound intensity, can be stored and/or retrieved from a database associated with the environment property, which can be the same database as the database associated with the user.

The process can increase or decrease the likelihood that the user is asleep based on the sleep state associated with the user, and the confidence level associated with the sleep state determination. For example, if the sleep state associated with the user is awake, the process decreases the likelihood that the user is asleep by an amount corresponding to the confidence level associated with the awake sleep state. For example, the process multiplies the likelihood that the user is asleep by (1—the confidence level). Similarly, if the sleep state associated with the user is asleep, the process increases the likelihood that the user is asleep by an amount corresponding to the confidence level associated with the sleep state. For example, the process multiplies the likelihood that the user is asleep by the confidence level.

Based on the likelihood that the user is asleep, the process, at block 1050, sends an appropriate control signal to an appliance. For example, if the user is asleep, the process sends the control signal to the thermostat to adjust the temperature to the average nightly temperature. Further, if the user is asleep and the lights are on, the process turns off the lights. Similarly, if the user is asleep and a media device is on, such as a television, a mobile device, a PlayStation, etc., the process turns off the media device. If the user is asleep, and the humidifier is off, the process sends a control signal to the humidifier to turn on. If the user is asleep, the process sends a control signal to the locks to engage. If the user is awake, the process sends the control signal to: the thermostat to adjust the temperature to the average temperature associated with the current time, the coffee maker to start making coffee, the humidifier to turn off, etc.

### Smart Home

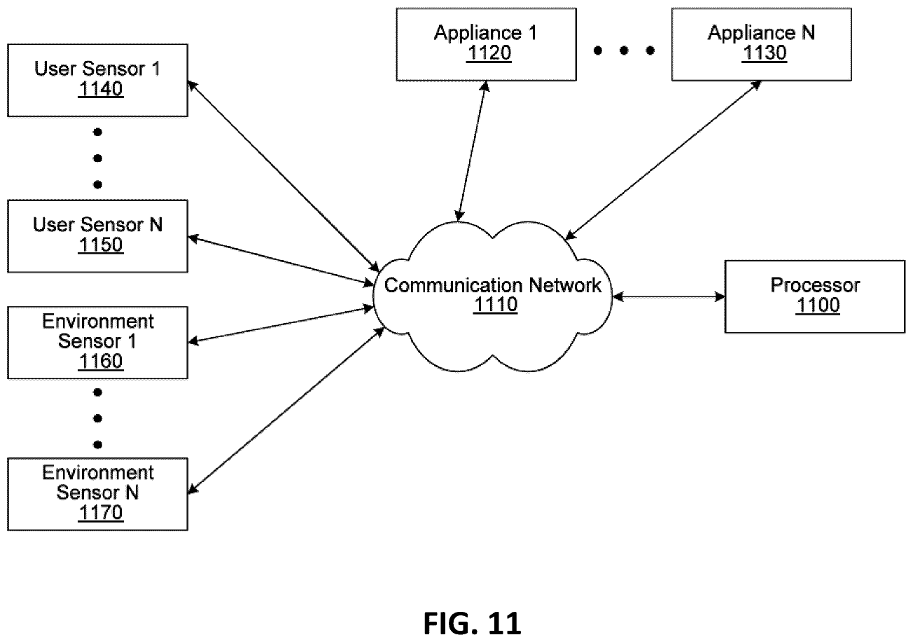

FIG. 11 is a diagram of a system capable of automating the control of the home appliances, according to one embodiment. Any number of user sensors 1140, 1150 monitor biological signals associated with said user, such as temperature, motion, presence, heart rate, or breathing rate. Any number of environment sensors 1160, 1170 monitor environment properties, such as temperature, sound, light, or humidity. According to one embodiment, the environment sensors 1160, 1170 are placed next to a bed. The user sensors 1140, 1150 and the environment sensors 1160, 1170 communicate their measurements to the processor 1100. The processor 1100 determines, based on the current biological signals associated with said user, historical biological signals associated with said user, user-specified preferences, exercise data associated with said user, and the environment properties received, a control signal, and a time to send said control signal to an appliance 1120, 1130.

The processor 1100 is any type of microcontroller, or any processor in a mobile terminal, fixed terminal, or portable terminal, including a mobile handset, station, unit, device, multimedia computer, multimedia tablet, Internet node, cloud computer, communicator, desktop computer, laptop computer, notebook computer, netbook computer, tablet computer, personal communication system (PCS) device, personal navigation device, personal digital assistants (PDAs), audio/video player, digital camera/camcorder, positioning device, television receiver, radio broadcast receiver, electronic book device, game device, the accessories and peripherals of these devices, or any combination thereof.

The processor 1100 can be connected to the user sensor 1140, 1150, or the environment sensor 1160, 1170 by a computer bus, such as an I2C bus. Also, the processor 1100 can be connected to the user sensor 1140, 1150, or environment sensor 1160, 1170 by a communication network 1110. By way of example, the communication network 1110 connecting the processor 1100 to the user sensor 1140, 1150, or the environment sensor 1160, 1170, includes one or more networks such as a data network, a wireless network, a telephony network, or any combination thereof. The data network may be any local area network (LAN), metropolitan area network (MAN), wide area network (WAN), public data network (e.g., the Internet), short range wireless network, or any other suitable packet-switched network, such as a commercially owned, proprietary packet-switched network, e.g., a proprietary cable or fiber-optic network, and the like, or any combination thereof. In addition, the wireless network may be, for example, a cellular network and may employ various technologies, including enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., worldwide interoperability for microwave access (WiMAX), Long Term Evolution (LTE) networks, code division multiple access (CDMA), wideband code division multiple access (WCDMA), wireless fidelity (WiFi), wireless LAN (WLAN), Bluetooth®, Internet Protocol (IP) data casting, satellite, mobile ad-hoc network (MANET), and the like, or any combination thereof.

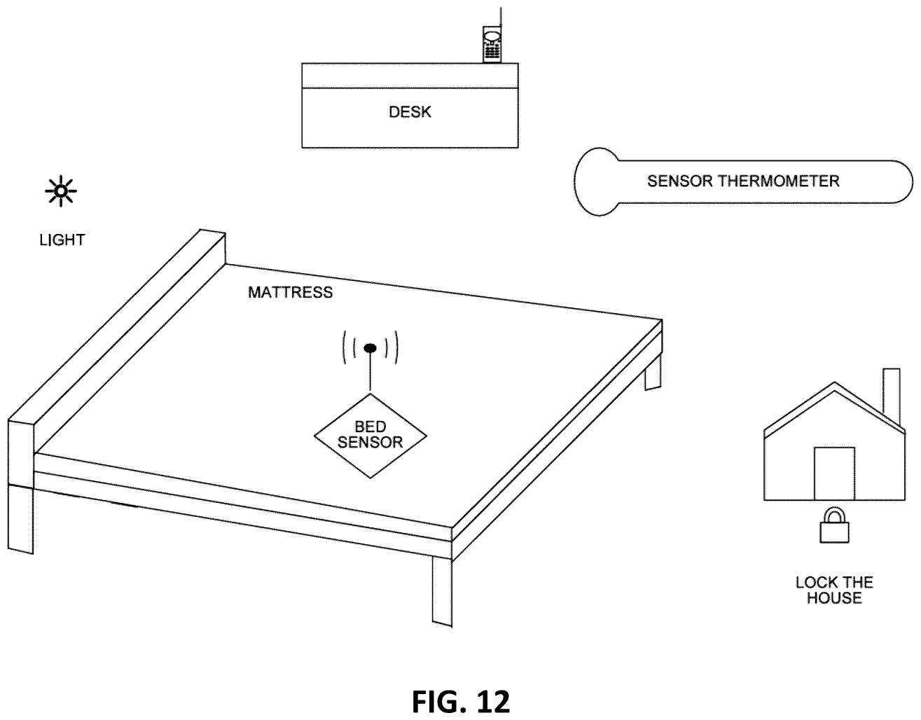

FIG. 12 is an illustration of the system capable of controlling an appliance and a home, according to one embodiment. The appliances, which the system disclosed here can control, comprise an alarm, a coffee machine, a lock, a thermostat, a bed device, a humidifier, or a light. For example, the system detects that the user has fallen asleep, the system sends a control signal to the lights to turn off, to the locks to engage, and to the thermostat to lower the temperature. According to another example, if the system detects that the user has woken up and it is morning, the system sends a control signal to the coffee machine to start making coffee.

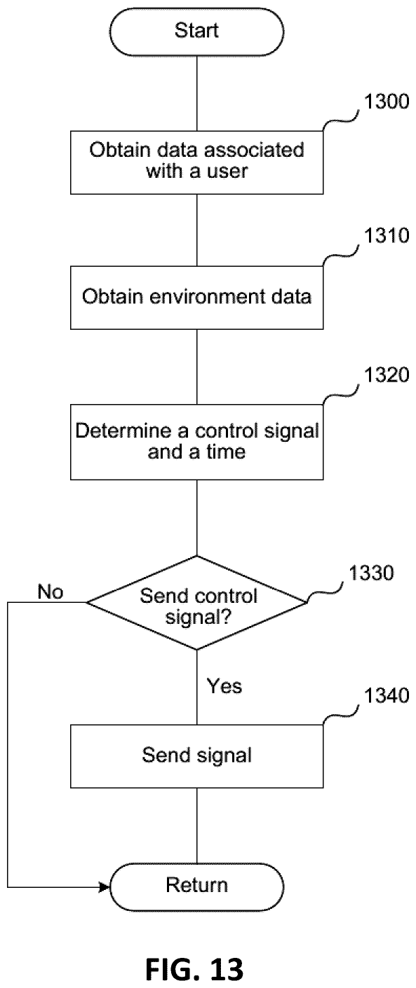

FIG. 13 is a flowchart of the process for controlling an appliance, according to one embodiment. In one embodiment, at block 1300, the process obtains history of biological signals, such as at what time the user goes to bed on a particular day of the week (e.g., the average bedtime associated with said user on Monday, the average bedtime associated with said user on Tuesday, etc.). The history of biological signals can be stored in a database associated with the user, or in a database associated with the bed device. In another embodiment, at block 1300, the process also obtains user specified preferences, such as the preferred bed temperature associated with said user. Based on the history of biological signals and user-specified preferences, the process, at block 1320, determines a control signal, and a time to send said control signal to an appliance. At block 1330, the process determines whether to send a control signal to an appliance. For example, if the current time is within half an hour of the average bedtime associated with said user on that particular day of the week, the process, at block 1340, sends a control signal to an appliance. For example, the control signal comprises an instruction to turn on the bed device, and the user specified bed temperature. Alternatively, the bed temperature is determined automatically, such as by calculating the average nightly bed temperature associated with a user.

According to another embodiment, at block 1300, the process obtains a current biological signal associated with a user from a sensor associated with said user. At block 1310, the process also obtains environment data, such as the ambient light, from an environment sensor associated with a bed device. Based on the current biological signal, the process identifies whether the user is asleep. If the user is asleep and the lights are on, the process sends an instruction to turn off the lights. In another embodiment, if the user is asleep, the lights are off, and the ambient light is high, the process sends an instruction to the blinds to shut. In another embodiment, if the user is asleep, the process sends an instruction to the locks to engage.

In another embodiment, the process, at block 1300, obtains a history of biological signals, such as at what time the user goes to bed on a particular day of the week (e.g., the average bedtime associated with said user on Monday, the average bedtime associated with said user on Tuesday, etc.). The history of biological signals can be stored in a database associated with the bed device, or in a database associated with a user. Alternatively, the user may specify a bedtime for the user for each day of the week. Further, the process obtains the exercise data associated with said user, such as the number of hours the user spent exercising, or the heart rate associated with said user during exercising. According to one embodiment, the process obtains the exercise data from a user phone, a wearable device, fitbit bracelet, or database associated with said user. Based on the average bedtime for that day of the week and the exercise data during the day, the process, at block 1320, determines the expected bedtime associated with said user that night. The process then sends an instruction to the bed device to heat to a desired temperature, before the expected bedtime. The desired temperature can be specified by the user, or the desired temperature can be determined automatically, based on the average nightly temperature associated with said user.

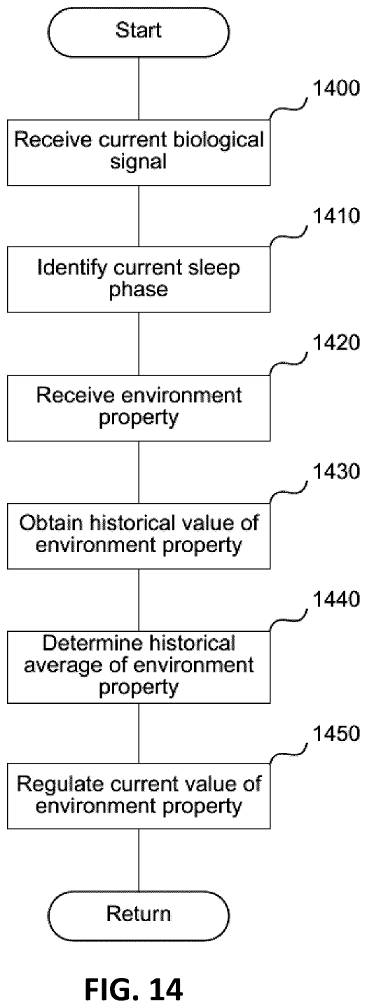

FIG. 14 is a flowchart of the process for controlling an appliance, according to another embodiment. The process, at block 1400, receives the current biological signal associated with said user, such as the heart rate, breathing rate, presence, motion, or temperature, associated with said user. Based on the current biological signal, the process, at block 1410, identifies current sleep phase, such as light sleep, deep sleep, or REM sleep. The process, at block 1420 also receives a current environment property value, such as the temperature, the humidity, the light, or the sound. The process, at block 1430, accesses a database, which stores historical values associated with the environment property and the current sleep phase. That is, the database associates each sleep phase with an average historical value of the different environment properties. The database may be associated with the bed device, may be associated with the user, or may be associated with a remote server. The process, at block 1440, then calculates a new average of the environment property based on the current value of the environment property and the historical value of the environment property, and assigns the new average to the current sleep phase in the database. If there is a mismatch between the current value of the environment property and the historical average, the process, at block 1450, regulates the current value to match the historical average. For example, the environment property can be the temperature associated with the bed device. The database stores the average bed temperature corresponding to each of the sleep phases, light sleep, deep sleep, REM sleep. If the current bed temperature is below the historical average, the process sends a control signal to increase the temperature of the bed to match the historical average.

### Monitoring of Biological Signals

Biological signals associated with a person, such as a heart rate or a breathing rate, indicate said person's state of health. Changes in the biological signals can indicate an immediate onset of a disease, or a long-term trend that increases the risk of a disease associated with said person. Monitoring the biological signals for such changes can predict the onset of a disease, can enable calling for help when the onset of the disease is immediate, or can provide advice to the person if the person is exposed to a higher risk of the disease in the long-term.

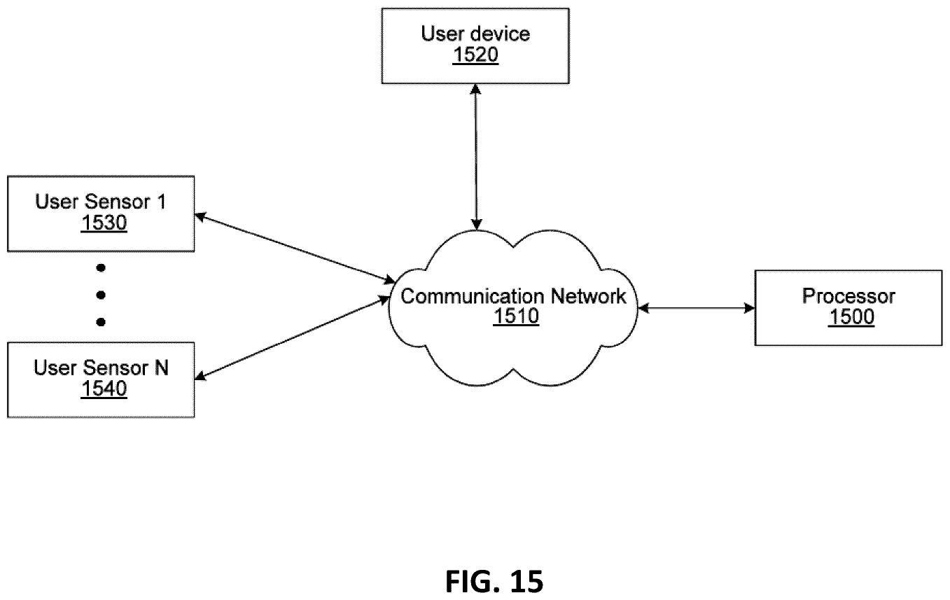

FIG. 15 is a diagram of a system for monitoring biological signals associated with a user, and providing notifications or alarms, according to one embodiment. Any number of user sensors 1530, 1540 monitor bio signals associated with said user, such as temperature, motion, presence, heart rate, or breathing rate. The user sensors 1530, 1540 communicate their measurements to the processor 1500. The processor 1500 determines, based on the bio signals associated with said user, historical biological signals associated with said user, or user-specified preferences, whether to send a notification or an alarm to a user device 1520. In some embodiments, the user device 1520 and the processor 1500 can be the same device.

The user device 1520 is any type of a mobile terminal, fixed terminal, or portable terminal, including a mobile handset, station, unit, device, multimedia computer, multimedia tablet, Internet node, communicator, desktop computer, laptop computer, notebook computer, netbook computer, tablet computer, personal communication system (PCS) device, personal navigation device, personal digital assistants (PDAs), audio/video player, digital camera/camcorder, positioning device, television receiver, radio broadcast receiver, electronic book device, game device, the accessories and peripherals of these devices, or any combination thereof.

The processor 1500 is any type of microcontroller, or any processor in a mobile terminal, fixed terminal, or portable terminal, including a mobile handset, station, unit, device, multimedia computer, multimedia tablet, Internet node, cloud computer, communicator, desktop computer, laptop computer, notebook computer, netbook computer, tablet computer, personal communication system (PCS) device, personal navigation device, personal digital assistants (PDAs), audio/video player, digital camera/camcorder, positioning device, television receiver, radio broadcast receiver, electronic book device, game device, the accessories and peripherals of these devices, or any combination thereof.

The processor 1500 can be connected to the user sensor 1530, 1540 by a computer bus, such as an I2C bus. Also, the processor 1500 can be connected to the user sensor 1530, 1540 by a communication network 1510. By way of example, the communication network 1510 connecting the processor 1500 to the user sensor 1530, 1540 includes one or more networks, such as a data network, a wireless network, a telephony network, or any combination thereof. The data network may be any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, or any combination thereof. In addition, the wireless network may be, for example, a cellular network and may employ various technologies, including enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., worldwide interoperability for microwave access (WiMAX), Long Term Evolution (LTE) networks, code division multiple access (CDMA), wideband code division multiple access (WCDMA), wireless fidelity (WiFi), wireless LAN (WLAN), Bluetooth®, Internet Protocol (IP) data casting, satellite, mobile ad-hoc network (MANET), and the like, or any combination thereof.

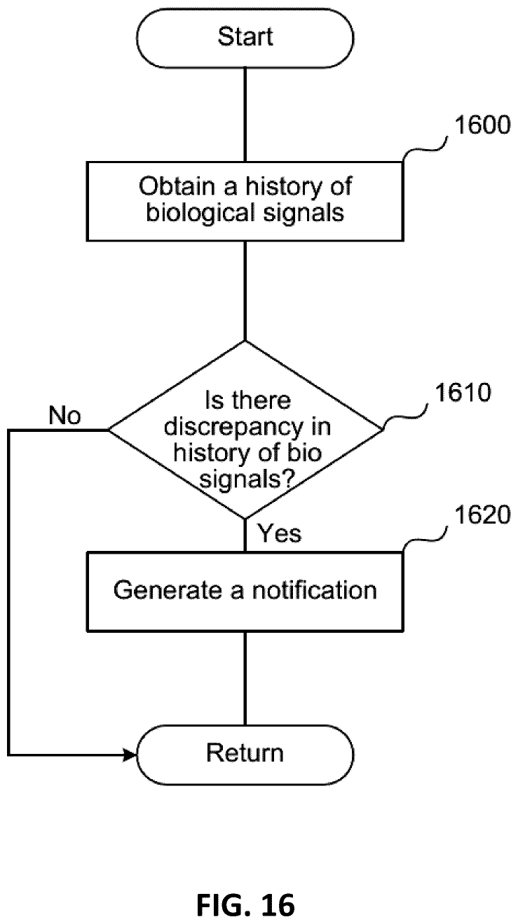

FIG. 16 is a flowchart of a process for generating a notification based on a history of biological signals associated with a user, according to one embodiment. The process, at block 1600, obtains a history of biological signals, such as the presence history, motion history, breathing rate history, or heart rate history, associated with said user. The history of biological signals can be stored in a database associated with a user. At block 1610, the process determines if there is an irregularity in the history of biological signals within a timeframe. If there is an irregularity, at block 1620, the process generates a notification to the user. The timeframe can be specified by the user, or the timeframe can be automatically determined based on the type of irregularity. For example, the heart rate associated with said user goes up within a one-day timeframe when the user is sick. According to one embodiment, the process detects an irregularity, specifically, that a daily heart rate associated with said user is higher than normal. Consequently, the process warns the user that the user may be getting sick. According to another embodiment, the process detects an irregularity, such as that an elderly user is spending at least 10% more time in bed per day over the last several days, than the historical average. The process generates a notification to the elderly user, or to the elderly user's caretaker, such as how much more time the elderly user is spending in bed. In another embodiment, the process detects an irregularity such as an increase in resting heart rate, by more than 15 beats per minute, over a ten-year period. Such an increase in the resting heart rate doubles the likelihood that the user will die from a heart disease, compared to those people whose heart rates remained stable. Consequently, the process warns the user that the user is at risk of a heart disease.

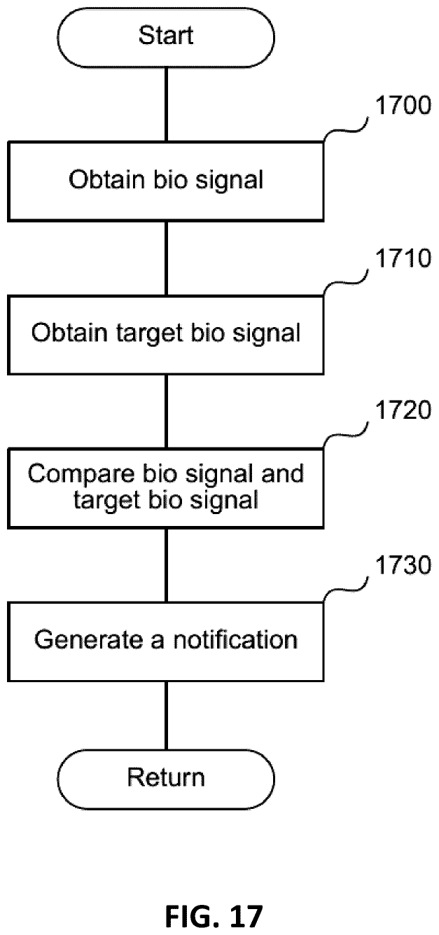

FIG. 17 is a flowchart of a process for generating a comparison between a biological signal associated with a user and a target biological signal, according to one embodiment. The process, at block 1700, obtains a current biological signal associated with a user, such as presence, motion, breathing rate, temperature, or heart rate, associated with said user. The process obtains said current biological signal from a sensor associated with said user. The process, at block 1710, then obtains a target biological signal, such as a user-specified biological signal, a biological signal associated with a healthy user, or a biological signal associated with an athlete. According to one embodiment, the process obtains said target biological signal from a user or a database storing biological signals. The process, at block 1720, compares the current bio signal associated with said user and the target bio signal. At block 1730, the process generates a notification based on the comparison. The comparison of the current bio signal associated with said user and the target bio signal comprises detecting a higher frequency in the current biological signal than in the target biological signal, detecting a lower frequency in the current biological signal than in the target biological signal, detecting higher amplitude in the current biological signal than in the target biological signal, or detecting lower amplitude in the current biological signal than in the target biological signal.

According to one embodiment, the process of FIG. 17 can be used to detect if an infant has a higher risk of sudden infant death syndrome (“SIDS”). In SIDS victims less than one month of age, heart rate is higher than in healthy infants of the same age, during all sleep phases. SIDS victims greater than one month of age show higher heart rates during the REM sleep phase. In case of monitoring an infant for a risk of SIDS, the process obtains the current bio signal associated with the sleeping infant, and a target biological signal associated with the heart rate of a healthy infant, where the heart rate is at the high end of a healthy heart rate spectrum. The process obtains the current bio signal from a user sensor associated with the sleeping infant. The process obtains said target biological signal from a database of biological signals. If the frequency of the biological signal of the infant exceeds the target biological signal, the process generates a notification to the infant's caretaker that the infant is at higher risk of SIDS.

According to another embodiment, the process of FIG. 17 can be used in fitness training. A normal resting heart rate for adults ranges from 60 to 100 beats per minute. Generally, a lower heart rate at rest implies more efficient heart function and better cardiovascular fitness. For example, a well-trained athlete might have a normal resting heart rate closer to 40 beats per minute. Thus, a user may specify a target rest heart rate of 40 beats per minute. The process of FIG. 17 generates a comparison between the actual bio signal associated with said user and the target bio signal, at block 1720, and, based on the comparison, the process, at block 1730, generates a notification whether the user has reached his target or whether the user needs to exercise more.

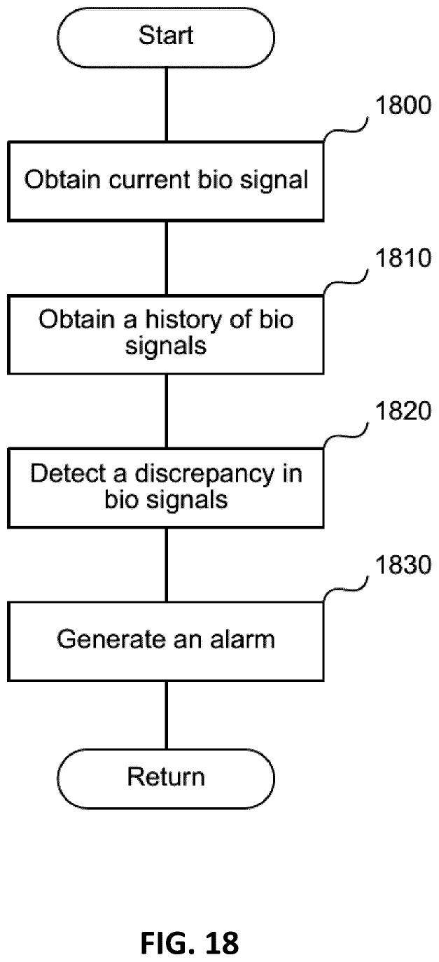

FIG. 18 is a flowchart of a process for detecting the onset of a disease, according to one embodiment. The process, at block 1800, obtains the current bio signal associated with a user, such as presence, motion, temperature, breathing rate, or heart rate, associated with said user. The process obtains the current bio signal from a sensor associated with said user. Further, the process, at block 1810, obtains a history of bio signals associated with said user from a database. The history of bio signals comprises the bio signals associated with said user, accumulated over time. The history of biological signals can be stored in a database associated with a user. The process, at block 1820, then detects a discrepancy between the current bio signal and the history of bio signals, where the discrepancy is indicative of an onset of a disease. The process, at block 1830, then generates an alarm to the user's caretaker. The discrepancy between the current bio signal and the history of bio signals comprises a higher frequency in the current bio signal than in the history of bio signals, or a lower frequency in the current bio signal than in the history of bio signals.

According to one embodiment, the process of FIG. 18 can be used to detect an onset of an epileptic seizure. A healthy person has a normal heart rate between 60 and 100 beats per minute. During epileptic seizures, the median heart rate associated with said person exceeds 100 beats per minute. The process of FIG. 18 detects that the heart rate associated with said user exceeds the normal heart rate range associated with said user. The process then generates an alarm to the user's caretaker that the user is having an epileptic seizure. Although rare, epileptic seizures can cause the median heart rate associated with a person to drop below 40 beats per minute. Similarly, the process of FIG. 18 detects if the current heart rate is below the normal heart rate range associated with said user. The process then generates an alarm to the user's caretaker that the user is having an epileptic seizure.

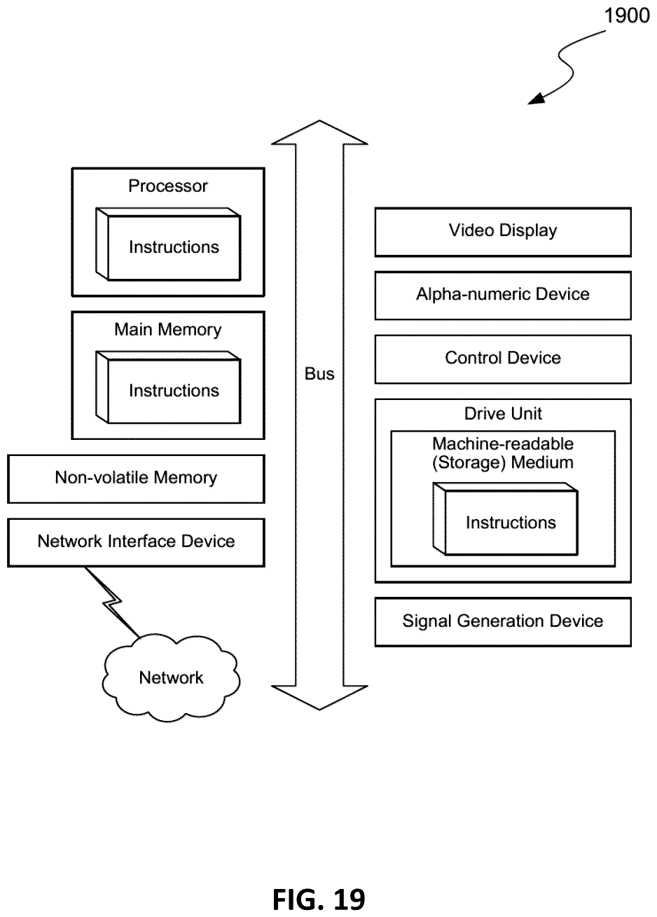

FIG. 19 is a diagrammatic representation of a machine in the example form of a computer system 1900 within which a set of instructions, for causing the machine to perform any one or more of the methodologies or modules discussed herein, may be executed.

In the example of FIG. 19, the computer system 1900 includes a processor, memory, non-volatile memory, and an interface device. Various common components (e.g., cache memory) are omitted for illustrative simplicity. The computer system 1900 is intended to illustrate a hardware device on which any of the components described in the example of FIGS. 1-18 (and any other components described in this specification) can be implemented. The computer system 1900 can be of any applicable known or convenient type. The components of the computer system 1900 can be coupled together via a bus or through some other known or convenient device.

This disclosure contemplates the computer system 1900 taking any suitable physical form. As an example and not by way of limitation, computer system 1900 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, or a combination of two or more of these. Where appropriate, computer system 1900 may include one or more computer systems 1900; be unitary or distributed; span multiple locations; span multiple machines; or reside in a cloud, which may include one or more cloud components in one or more networks. Where appropriate, one or more computer systems 1900 may perform, without substantial spatial or temporal limitation, one or more steps of one or more methods described or illustrated herein. As an example and not by way of limitation, one or more computer systems 1900 may perform in real time or in batch mode one or more steps of one or more methods described or illustrated herein. One or more computer systems 1900 may perform at different times or at different locations one or more steps of one or more methods described or illustrated herein, where appropriate.

The processor may be, for example, a conventional microprocessor such as an Intel Pentium microprocessor or Motorola power PC microprocessor. One of skill in the relevant art will recognize that the terms “machine-readable (storage) medium” or “computer-readable (storage) medium” include any type of device that is accessible by the processor.

The memory is coupled to the processor by, for example, a bus. The memory can include, by way of example but not limitation, random access memory (RAM), such as dynamic RAM (DRAM) and static RAM (SRAM). The memory can be local, remote, or distributed.

The bus also couples the processor to the non-volatile memory and drive unit. The non-volatile memory is often a magnetic floppy or hard disk, a magnetic-optical disk, an optical disk, a read-only memory (ROM), such as a CD-ROM, EPROM, or EEPROM, a magnetic or optical card, or another form of storage for large amounts of data. Some of this data is often written, by a direct memory access process, into memory during execution of software in the computer 1900. The non-volatile storage can be local, remote, or distributed. The non-volatile memory is optional because systems can be created with all applicable data available in memory. A typical computer system will usually include at least a processor, memory, and a device (e.g., a bus) coupling the memory to the processor.

Software is typically stored in the non-volatile memory and/or the drive unit. Indeed, storing an entire large program in memory may not even be possible. Nevertheless, it should be understood that, for software to run, if necessary, it is moved to a computer readable location appropriate for processing, and for illustrative purposes, that location is referred to as the memory in this paper. Even when software is moved to the memory for execution, the processor will typically make use of hardware registers to store values associated with the software, and local cache that, ideally, serves to speed up execution. As used herein, a software program is assumed to be stored at any known or convenient location (from non-volatile storage to hardware registers) when the software program is referred to as “implemented in a computer-readable medium.” A processor is considered to be “configured to execute a program” when at least one value associated with the program is stored in a register readable by the processor.

The bus also couples the processor to the network interface device. The interface can include one or more of a modem or network interface. It will be appreciated that a modem or network interface can be considered to be part of the computer system 1900. The interface can include an analog modem, ISDN modem, cable modem, token ring interface, satellite transmission interface (e.g., “direct PC”), or other interfaces for coupling a computer system to other computer systems. The interface can include one or more input and/or output devices. The I/O devices can include, by way of example but not limitation, a keyboard, a mouse or other pointing device, disk drives, printers, a scanner, and other input and/or output devices, including a display device. The display device can include, by way of example but not limitation, a cathode ray tube (CRT), liquid crystal display (LCD), or some other applicable known or convenient display device. For simplicity, it is assumed that controllers of any devices not depicted in the example of FIG. 9 reside in the interface.

In operation, the computer system 1900 can be controlled by operating system software that includes a file management system, such as a disk operating system. One example of operating system software with associated file management system software is the family of operating systems known as Windows® from Microsoft Corporation of Redmond, Washington, and their associated file management systems. Another example of operating system software with its associated file management system software is the Linux™ operating system and its associated file management system. The file management system is typically stored in the non-volatile memory and/or drive unit and causes the processor to execute the various acts required by the operating system to input and output data and to store data in the memory, including storing files on the non-volatile memory and/or drive unit.

Some portions of the detailed description may be presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, as apparent from the following discussion, it is appreciated that, throughout the description, discussions utilizing terms such as “processing” or “computing” or “calculating” or “determining” or “displaying” or “generating” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The algorithms and displays presented herein are not inherently related to any particular computer or other apparatus. Various general purpose systems may be used with programs in accordance with the teachings herein, or it may prove convenient to construct more specialized apparatus to perform the methods of some embodiments. The required structure for a variety of these systems will appear from the description below. In addition, the techniques are not described with reference to any particular programming language, and various embodiments may thus be implemented using a variety of programming languages.

In alternative embodiments, the machine operates as a standalone device or may be connected (e.g., networked) to other machines. In a networked deployment, the machine may operate in the capacity of a server or a client machine in a client-server network environment, or as a peer machine in a peer-to-peer (or distributed) network environment.

The machine may be a server computer, a client computer, a personal computer (PC), a tablet PC, a laptop computer, a set-top box (STB), a personal digital assistant (PDA), a cellular telephone, an iPhone, a Blackberry, a processor, a telephone, a web appliance, a network router, switch or bridge, or any machine capable of executing a set of instructions (sequential or otherwise) that specify actions to be taken by that machine.

While the machine-readable medium or machine-readable storage medium is shown in an exemplary embodiment to be a single medium, the term “machine-readable medium” and “machine-readable storage medium” should be taken to include a single medium or multiple media (e.g., a centralized or distributed database, and/or associated caches and servers) that store the one or more sets of instructions. The term “machine-readable medium” and “machine-readable storage medium” shall also be taken to include any medium that is capable of storing, encoding or carrying a set of instructions for execution by the machine and that cause the machine to perform any one or more of the methodologies or modules of the presently disclosed technique and innovation.

In general, the routines executed to implement the embodiments of the disclosure may be implemented as part of an operating system or a specific application, component, program, object, module or sequence of instructions referred to as “computer programs.” The computer programs typically comprise one or more instructions set at various times in various memory and storage devices in a computer, and that, when read and executed by one or more processing units or processors in a computer, cause the computer to perform operations to execute elements involving the various aspects of the disclosure.

Moreover, while embodiments have been described in the context of fully functioning computers and computer systems, those skilled in the art will appreciate that the various embodiments are capable of being distributed as a program product in a variety of forms, and that the disclosure applies equally regardless of the particular type of machine or computer-readable media used to actually effect the distribution.

Further examples of machine-readable storage media, machine-readable media, or computer-readable (storage) media include but are not limited to recordable type media such as volatile and non-volatile memory devices, floppy and other removable disks, hard disk drives, optical disks (e.g., Compact Disk Read-Only Memory (CD ROMS), Digital Versatile Disks, (DVDs), etc.), among others, and transmission type media, such as digital and analog communication links.

In some circumstances, operation of a memory device, such as a change in state from a binary one to a binary zero or vice-versa, for example, may comprise a transformation, such as a physical transformation. With particular types of memory devices, such a physical transformation may comprise a physical transformation of an article to a different state or thing. For example, but without limitation, for some types of memory devices, a change in state may involve an accumulation and storage of charge or a release of stored charge. Likewise, in other memory devices, a change of state may comprise a physical change or transformation in magnetic orientation or a physical change or transformation in molecular structure, such as from crystalline to amorphous or vice versa. The foregoing is not intended to be an exhaustive list of all examples in which a change in state for a binary one to a binary zero or vice-versa in a memory device may comprise a transformation, such as a physical transformation. Rather, the foregoing is intended as illustrative examples.

A storage medium typically may be non-transitory or comprise a non-transitory device. In this context, a non-transitory storage medium may include a device that is tangible, meaning that the device has a concrete physical form, although the device may change its physical state. Thus, for example, non-transitory refers to a device remaining tangible despite this change in state.

### Remarks

In many of the embodiments disclosed in this application, the technology is capable of allowing multiple different users to use the same piece of furniture equipped with the presently disclosed technology. For example, different people can sleep in the same bed. In addition, two different users can switch the side of the bed that they sleep on, and the technology disclosed here will correctly identify which user is sleeping on which side of the bed. The technology identifies the users and obtains the user ID, based on any of the following signals alone or in combination: heart rate, breathing rate, body motion, or body temperature associated with each user. In another embodiment, the technology disclosed here identifies the user by receiving both the user ID and side of the bed associated with the user ID, from a device associated with the user.

The foregoing description of various embodiments of the claimed subject matter has been provided for the purposes of illustration and description. It is not intended to be exhaustive or to limit the claimed subject matter to the precise forms disclosed. Many modifications and variations will be apparent to one skilled in the art. Embodiments were chosen and described in order to best describe the principles of the invention and its practical applications, thereby enabling others skilled in the relevant art to understand the claimed subject matter, the various embodiments, and the various modifications that are suited to the particular uses contemplated.

While embodiments have been described in the context of fully functioning computers and computer systems, those skilled in the art will appreciate that the various embodiments are capable of being distributed as a program product in a variety of forms, and that the disclosure applies equally regardless of the particular type of machine or computer-readable media used to actually effect the distribution.

Although the above Detailed Description describes certain embodiments and the best mode contemplated, no matter how detailed the above appears in text, the embodiments can be practiced in many ways. Details of the systems and methods may vary considerably in their implementation details, while still being encompassed by the specification. As noted above, particular terminology used when describing certain features or aspects of various embodiments should not be taken to imply that the terminology is being redefined herein to be restricted to any specific characteristics, features, or aspects of the invention with which that terminology is associated. In general, the terms used in the following claims should not be construed to limit the invention to the specific embodiments disclosed in the specification, unless those terms are explicitly defined herein. Accordingly, the actual scope of the invention encompasses not only the disclosed embodiments, but also all equivalent ways of practicing or implementing the embodiments under the claims.

The language used in the specification has been principally selected for readability and instructional purposes, and it may not have been selected to delineate or circumscribe the inventive subject matter. It is therefore intended that the scope of the invention be limited not by this Detailed Description, but rather by any claims that issue on an application based hereon. Accordingly, the disclosure of various embodiments is intended to be illustrative, but not limiting, of the scope of the embodiments, which is set forth in the following claims.

### EMBODIMENTS

Embodiment 1. A bed system for automatically heating a mattress comprising: a bed pad device configured to receive an instruction to heat said mattress to a specified temperature, said bed pad device comprising a mattress cover, said mattress cover further comprising: a sensor strip, wherein said sensor strip comprises a user sensor configured to measure a biological signal associated with a user, wherein said biological signal comprises a heart rate associated with said user, and a breathing rate associated with said user; and a coil configured to heat said mattress; and an environment sensor, configured to measure an environment property, wherein said environment property comprises a temperature, a humidity, a light intensity, or a sound; and a computer processor communicatively coupled to said bed pad device and said environment sensor, said computer processor configured to: determine, based on said biological signal associated with said user and said environment property, a control signal, and a time to send said control signal; and send said control signal at said time to said bed pad device.

Embodiment 2. The bed system of embodiment 1, wherein said computer processor determines presence associated with said user, and motion associated with said user, based on said biological signal associated with said user.

Embodiment 3. The bed system of embodiment 1, wherein said computer processor is configured to receive an input from said user, and wherein said computer processor is configured to determine at least one of said control signal, or said time, based on said input from said user.

Embodiment 4. The bed system of embodiment 1, further comprising: a fabric, a batting, or a waterproof material.

Embodiment 5. The bed system of embodiment 1, wherein said time comprises an average bedtime associated with said user.

Embodiment 6. The bed system of embodiment 1, wherein said control signal comprises an instruction to heat said coil to a high temperature, wherein said high temperature comprises an average high temperature associated with said user.

Embodiment 7. A bed system comprising: a user sensor configured to measure a biological signal associated with a user; and an environment sensor, configured to measure an environment property; and a bed device configured to receive a control signal; and a computer processor communicatively coupled to said user sensor and said environment sensor, and further communicatively coupled to said bed device, said computer processor configured to: determine, based on said biological signal associated with said user and said environment property, the control signal, and a time to send said control signal to said bed device; and send said control signal at said time to said bed device.

Embodiment 8. The bed system of embodiment 7, wherein said bed device comprises a coil.

Embodiment 9. The bed system of embodiment 8 further comprising: a fabric, a batting, or a waterproof material.

Embodiment 10. The bed system of embodiment 9, wherein said batting comprises a sensor strip, wherein said sensor strip comprises said user sensor and a computer bus.

Embodiment 11. The bed system of embodiment 7, wherein said time comprises an average daily bedtime associated with said user.

Embodiment 12. The bed system of embodiment 7, wherein said biological signal corresponds to at least one of: a presence associated with said user, a motion associated with said user, a temperature associated with said user, a breathing rate associated with said user, or a heart rate associated with said user.

Embodiment 13. The bed system of embodiment 7, wherein said user sensor comprises at least one of a piezo sensor, or a temperature sensor.

Embodiment 14. The bed system of embodiment 7, wherein said control signal comprises an instruction to heat said bed device to a high temperature, wherein said high temperature is determined based on said biological signal associated with said user.

Embodiment 15. The bed system of embodiment 7, wherein said control signal comprises an instruction to cool said bed device to a low temperature, wherein said low temperature is determined based on said biological signal associated with said user.

Embodiment 16. The bed system of embodiment 7, wherein said environment property comprises temperature, humidity, light, or sound.

Embodiment 17. The bed system of embodiment 7, wherein said computer processor is configured to receive input from said user, whereby said computer processor, based on said input from said user, determines said time to send said control signal to said bed device.

Embodiment 18. A system comprising: a user sensor configured to measure a biological signal associated with a user; and an environment sensor, configured to measure an environment property; and a transceiver communicatively coupled to said user sensor and said environment sensor, said transceiver configured to transmit said biological signal associated with said user and said environment property, and further said transceiver configured to receive a control signal; and a bed device, communicatively coupled to said transceiver, wherein said bed device is configured to respond to said control signal.

Embodiment 19. The system of embodiment 18, wherein said biological signal associated with said user corresponds to at least one of a temperature associated with said user, a presence associated with said user, a motion associated with said user, a breathing rate associated with said user, or a heart rate associated with said user.

Embodiment 20. The system of embodiment 18, wherein said environment property comprises a temperature, a humidity, a light intensity, or a sound.

Embodiment 21. The system of embodiment 18, wherein said control signal comprises an instruction to heat said bed device to a high temperature, wherein said high temperature comprises an average high temperature associated with said user.

Embodiment 22. The system of embodiment 18, wherein said control signal comprises an instruction to cool said bed device to a low temperature, wherein said low temperature comprises an average low temperature associated with said user.

Embodiment 23. A method comprising: (a) obtaining a biological signal associated with a user from a sensor; (b) obtaining a history of biological signals associated with said user from a database associated with said user; (c) obtaining an environment property from an environment sensor associated with a bed device; (d) determining, based on said biological signal associated with said user, said history of biological signals associated with said user, and said environment property, a control signal, and a time to send said control signal to said bed device; and (e) sending said control signal at said time to said bed device.

Embodiment 24. The method of embodiment 23, wherein obtaining said history of biological signals comprises: obtaining a plurality of average bedtimes associated with said user, wherein said plurality of average bedtimes comprises average bedtime on one or more days of a week.

Embodiment 25. The method of embodiment 23, wherein obtaining said biological signal comprises: obtaining at least one of: a presence associated with said user, a motion associated with said user, a breathing rate associated with said user, a temperature associated with said user, or a heart rate associated with said user.

Embodiment 26. The method of embodiment 23, wherein sending said control signal comprises: sending an instruction to heat said bed device to a high temperature, wherein said high temperature comprises an average high temperature associated with said user.

Embodiment 27. The method of embodiment 23, wherein sending said control signal comprises: sending an instruction to cool said bed device to a low temperature, wherein said low temperature comprises an average low temperature associated with said user.

Embodiment 28. The method of embodiment 23, wherein obtaining said environment property comprises: obtaining a temperature, a humidity, a light intensity, or a sound.

Embodiment 29. The method of embodiment 23, wherein determining said time to send said control signal to said bed device comprises calculating an average daily bedtime associated with said user.

Embodiment 30. A method comprising: (a) obtaining a compound biological signal associated with a user from a sensor; (b) extracting a heart rate signal from said compound biological signal; (c) extracting a breathing rate signal from said compound biological signal; (d) obtaining a wake-up time specified by said user; (e) determining sleep phase associated with said user based on said heart rate signal, and said breathing rate signal; and (f) activating an alarm based on said sleep phase and said wake-up time specified by said user.

Embodiment 31. The method of embodiment 30, wherein activating the alarm comprises: checking that said sleep phase is light sleep; and checking that current time is at most one hour before said wake-up time specified by said user.

Embodiment 32. The method of embodiment 30, wherein said alarm comprises a vibrating alarm attached to a cover, or a vibrating alarm attached to a mattress associated with said user.

Embodiment 33. The method of embodiment 30, wherein said alarm comprises a wearable alarm.

Embodiment 34. The method of embodiment 30, further comprising storing said heart rate signal and said breathing rate signal associated with said sleep phase associated with said user in a database.

Embodiment 35. The method of embodiment 30, further comprising identifying said user based on at least one of: said heart rate signal, or said breathing rate signal.

Embodiment 36. The method of embodiment 30, wherein said sleep phase comprises light sleep, deep sleep, or REM sleep.

Embodiment 37. The method of embodiment 30, wherein extracting said breathing rate signal from said compound biological signal comprises performing low-pass filtering of said compound biological signal.

Embodiment 38. The method of embodiment 30, wherein extracting said heart rate signal from said compound biological signal comprises performing bandpass filtering of said compound biological signal.

Embodiment 39. A method comprising: (a) obtaining a compound biological signal associated with a user; (b) extracting a heart rate signal from said compound biological signal; (c) extracting a breathing rate signal from said compound biological signal and; (d) obtaining an environment property; (e) determining a sleep phase associated with said user based on said heart rate signal, said breathing rate signal, and said environment property; and (f) determining a time to turn off an appliance based on said sleep phase and said environment property.

Embodiment 40. The method of embodiment 39, wherein determining the time to turn off said appliance comprises determining that said user is asleep.

Embodiment 41. The method of embodiment 39, wherein said appliance comprises a light, an entertainment device, or a thermostat.

Embodiment 42. The method of embodiment 39, further comprising identifying said user based on at least one of: said heart rate signal, or said breathing rate signal.

Embodiment 43. The method of embodiment 39, wherein said sleep phase comprises a light sleep, a deep sleep, or a REM sleep.

Embodiment 44. The method of embodiment 39, wherein said environment property comprises a temperature, a humidity, a light intensity, a sound, or a current time.

Embodiment 45. The method of embodiment 39, wherein extracting said breathing rate signal from said compound biological signal comprises performing low-pass filtering of said compound biological signal.

Embodiment 46. The method of embodiment 39, wherein extracting said heart rate signal from said compound biological signal comprises performing bandpass filtering of said compound biological signal.

Embodiment 47. A method comprising: (a) obtaining a history of sleep phase information associated with a user from a database; (b) obtaining a wake-up time specified by said user; (c) obtaining exercise information associated with said user; and (d) recommending a bedtime to said user based on said history of sleep phase information, said wake-up time specified by said user, and said exercise information associated with said user.

Embodiment 48. The method of embodiment 47, wherein said exercise information comprises daily exercise information associated with said user.

Embodiment 49. The method of embodiment 47, wherein said history of sleep phase information comprises an amount of time said user spent in light sleep during each day of a week, an amount of time said user spent in deep sleep during each day of a week, or an amount of time said user spent in REM sleep during each day of a week.

Embodiment 50. The method of embodiment 47, wherein said history of sleep phase information comprises a plurality of average bedtimes associated with said user, wherein each average bedtime of said plurality of average bedtimes corresponds to a day of a week.

Embodiment 51. A system comprising: a storage medium, communicatively coupled to a computer processor; and said computer processor, said computer processor configured to: obtain a history of sleep phase information associated with a user from a database; and obtain a wake-up time specified by said user; and obtain exercise information associated with said user; and recommend a bedtime for said user based on said history of sleep phase information, said wake-up time specified by said user, and said exercise information associated with said user.

Embodiment 52. The system of embodiment 51, wherein said exercise information comprises daily exercise information associated with said user.

Embodiment 53. The system of embodiment 51, wherein said history of sleep phase information comprises an amount of time said user spent in light sleep during each day of a week, an amount of time said user spent in deep sleep during each day of a week, or an amount of time said user spent in REM sleep during each day of a week.

Embodiment 54. The system of embodiment 51, wherein said history of sleep phase information comprises a plurality of average bedtimes associated with said user, wherein each average bedtime of said plurality of average bedtimes corresponds to a day of a week.

Embodiment 55. A method for automatically heating or cooling a mattress based on a biological signal associated with a user: (a) obtaining, by a processor, said biological signal associated with said user, wherein said biological signal comprises a respiration rate associated with said user, a heart rate associated with said user, a motion associated with said user, or a temperature associated with said user; (b) identifying, by said processor, said user based on at least one of: said heart rate associated with said user, said respiration rate associated with said user, said motion associated with said user, or said temperature associated with said user; (c) based on said user identification, obtaining, by said processor, from a database a normal biological signal range associated with a sleep phase in a plurality of sleep phases associated with said user, wherein said normal biological signal range comprises a normal temperature range associated with said user; (d) based on said normal biological signal range and said biological signal, identifying, by said processor, said sleep phase in said plurality of sleep phases associated with said user; and (e) when said temperature associated with said sleep phase is outside of said normal temperature range associated with said sleep phase, sending, by said processor, a control signal to a temperature control device coupled to said mattress, said control signal comprising an instruction to heat or cool said mattress to a temperature within said normal temperature range.

Embodiment 56. The method of embodiment 55, wherein said obtaining said biological signal comprises: measuring, by a sensor strip coupled to said mattress, said biological signal associated with said user, wherein said sensor strip comprises a piezo sensor, and a temperature sensor.

Embodiment 57. The method of embodiment 55, wherein said obtaining said biological signal comprises: receiving, by said processor, said biological signal associated with said user from a wearable sensor coupled to said user.

Embodiment 58. The method of embodiment 55, wherein said sending said control signal to said temperature control device comprises: determining, by said processor, a current time; based on said user identification, obtaining, by said processor, a wake-up time associated with said user; and when said current time is at most 3 hours before said wake-up time, sending by said processor, said control signal to said temperature control device coupled to said mattress, said control signal comprising an instruction to turn off.

Embodiment 59. The method of embodiment 55, wherein said identifying said sleep phase comprises detecting, by said processor, a slowdown in said heart rate, a drop in said temperature, and a regular respiration rate.

Embodiment 60. The method of embodiment 55, wherein said identifying said sleep phase comprises detecting, by said processor, an end of a preceding sleep phase in said plurality of sleep phases.

Embodiment 61. The method of embodiment 55, further comprising: obtaining, by said processor, perspiration associated with said user; and when said user is perspiring, sending, by said processor, said control signal comprising an instruction to cool said temperature control device until said user stops perspiring.

Embodiment 62. The method of embodiment 55, further comprising: obtaining, by said processor, perspiration associated with said user; and based on said perspiration, when said user wakes up, recommending, by said processor, an amount of liquid to consume.

Embodiment 63. A method for automatically heating or cooling a mattress based on a biological signal associated with a user: obtaining, by a processor, said biological signal associated with said user, wherein said biological signal comprises a respiration rate associated with said user, a heart rate associated with said user, a motion associated with said user, or a temperature associated with said user; based on said biological signal, detecting, by said processor, when said user has transitioned to sleep; and when said user has transitioned to sleep, sending, by said processor, a control signal to a temperature control device coupled to said mattress, said control signal comprising an instruction to cool said mattress to a predetermined temperature.

Embodiment 64. The method of embodiment 63, further comprising: identifying, by said processor, said user based on at least one of: said heart rate associated with said user, said respiration rate associated with said user, said motion associated with said user, or said temperature associated with said user; determining, by said processor, a current time; based on said user identification, obtaining, by said processor, a wake-up time associated with said user; and when said current time is at most 3 hours before said wake-up time, sending, by said processor, said control signal to said temperature control device coupled to said mattress, said control signal comprising an instruction to turn off.

Embodiment 65. The method of embodiment 63, further comprising: obtaining, by said processor, an ambient temperature surrounding said user; and when said ambient temperature is outside of a 35° C. to 36° C. range, sending, by said processor, said control signal to said temperature control device coupled to said mattress, said control signal comprising an instruction to cool or heat said mattress to a temperature within 27° C. to 35° C. range.

Embodiment 66. The method of embodiment 63, further comprising: identifying, by said processor, said user based on at least one of: said heart rate associated with said user, said respiration rate associated with said user, said temperature associated with said user, or said motion associated with said user; and based on said user identification, determining, by said processor, an average bedtime associated with said user; at said average bedtime associated with said user, sending, by said processor, said control signal to said temperature control device coupled to said mattress, wherein said control signal comprises one of an instruction to heat said temperature control device to a temperature in a 27° C. to 35° C. range, or an instruction to cool said temperature control device to said temperature in said 27° C. to 35° C. range.

Embodiment 67. The method of embodiment 63, wherein said obtaining said biological signal comprises: measuring, by a sensor strip coupled to said mattress, said biological signal associated with said user, wherein said sensor strip comprises a piezo sensor, and a temperature sensor.

Embodiment 68. The method of embodiment 63, wherein said obtaining said biological signal comprises: receiving, by said processor, said biological signal associated with said user from a wearable sensor coupled to said user.

Embodiment 69. The method of embodiment 63, wherein said sending said control signal to said temperature control device comprises: determining, by said processor, a current time; obtaining, by said processor, a wake-up time associated with said user; and when said current time is at most 3 hours before said wake-up time, sending, by said processor, said control signal to said temperature control device coupled to said mattress, said control signal comprising an instruction to turn off.

Embodiment 70. The method of embodiment 63, wherein said detecting when said user has transitioned to sleep comprises detecting, by said processor a drop in said temperature, or a regular respiration rate.

Embodiment 71. The method of embodiment 63, wherein said predetermined temperature is in a comfortable temperature range associated with a user.

Embodiment 72. A system for automatically heating or cooling a mattress based on a biological signal associated with a user: a processor; a storage medium storing computer-executable instructions that, when executed by said processor, cause the system to perform a computer-implemented operation, said instructions comprising: instructions for obtaining said biological signal associated with said user, wherein said biological signal comprises a respiration rate associated with said user, a heart rate associated with said user, a motion associated with said user, or a temperature associated with said user; instructions for identifying said user based on at least one of: said heart rate associated with said user, said respiration rate associated with said user, said motion associated with said user, or said temperature associated with said user; instructions for based on said user identification, obtaining from a database a normal biological signal range associated with a sleep phase in a plurality of sleep phases associated with said user, wherein said normal biological signal range comprises a normal temperature range associated with said user; instructions for based on said normal biological signal range and said biological signal, identifying said sleep phase in said plurality of sleep phases associated with said user; and instructions for when said temperature associated with said sleep phase is outside of said normal temperature range associated with said sleep phase, sending a control signal to a temperature control device coupled to said mattress, said control signal comprising an instruction to heat or cool said mattress to a temperature within said normal temperature range.

Embodiment 73. The system of embodiment 72, further comprising: a sensor strip coupled to said mattress, and to said processor, said sensor strip configured to measure said biological signal associated with said user, wherein said sensor strip comprises a piezo sensor, and a temperature sensor.

Embodiment 74. The system of embodiment 72, further comprising: a wearable sensor coupled to said user, and to said processor, said wearable sensor configured to measure said biological signal associated with said user.

Embodiment 75. The system of embodiment 72, wherein said instructions for sending said control signal to said temperature control device comprise: instructions for determining a current time; instructions for based on said user identification, obtaining a wake-up time associated with said user; and instructions for when said current time is at most 3 hours before said wake-up time, sending said control signal to said temperature control device coupled to said mattress, said control signal comprising an instruction to turn off.

Embodiment 76. The system of embodiment 72, wherein said instructions for identifying said sleep phase comprise instructions for detecting a slowdown in said heart rate, a drop in said temperature, and a regular respiration rate.

Embodiment 77. The system of embodiment 72, wherein said instructions for identifying said sleep phase comprise instructions for detecting an end of a preceding sleep phase in said plurality of sleep phases.

Embodiment 78. A system for automatically heating or cooling a mattress based on a biological signal associated with a user: a processor; a storage medium storing computer-executable instructions that, when executed by said processor, cause the system to perform a computer-implemented operation, said instructions comprising: instructions for obtaining said biological signal associated with said user, wherein said biological signal comprises a respiration rate associated with said user, a heart rate associated with said user, a motion associated with said user, or a temperature associated with said user; instructions for based on said biological signal, detecting when said user has transitioned to sleep; and instructions for when said user has transitioned to sleep, sending a control signal to a temperature control device coupled to said mattress, said control signal comprising an instruction to cool said mattress to a predetermined temperature.

Embodiment 79. The system of embodiment 78, said instructions further comprising: instructions for identifying said user based on at least one of: said heart rate associated with said user, said respiration rate associated with said user, said motion associated with said user, or said temperature associated with said user; instructions for determining a current time; instructions for based on said user identification, obtaining a wake-up time associated with said user; and instructions for when said current time is at most 3 hours before said wake-up time, sending said control signal to said temperature control device coupled to said mattress, said control signal comprising an instruction to turn off.

Embodiment 80. The system of embodiment 78, said instructions further comprising: instructions for obtaining an ambient temperature surrounding said user; and instructions for when said ambient temperature is outside of a 35° C. to 36° C. range, sending said control signal to said temperature control device coupled to said mattress, said control signal comprising an instruction to cool or heat said mattress to a temperature within 27° C. to 35° C. range.

Embodiment 81. The system of embodiment 78, said instructions further comprising: instructions for identifying said user based on at least one of: said heart rate associated with said user, said respiration rate associated with said user, said temperature associated with said user, or said motion associated with said user; and instructions for based on said user identification, determining an average bedtime associated with said user; instructions for at said average bedtime associated with said user, sending, said control signal to said temperature control device coupled to said mattress, wherein said control signal comprises one of an instruction to heat said temperature control device to a temperature in a 27° C. to 35° C. range, or an instruction to cool said temperature control device to said temperature in said 27° C. to 35° C. range.

Embodiment 82. A system of embodiment 78, further comprising: a sensor strip coupled to said mattress, said sensor strip configured to measure said biological signal associated with said user, wherein said sensor strip comprises a piezo sensor, and a temperature sensor.

Embodiment 83. A system of embodiment 78, further comprising: a wearable sensor coupled to said user and to said processor, said wearable sensor configured to measure said biological signal associated with said user.

Embodiment 84. A system of embodiment 78, wherein said instructions for sending said control signal to said temperature control device comprise: instructions for determining a current time; instructions for obtaining a wake-up time associated with said user; and instructions for when said current time is at most 3 hours before said wake-up time, sending said control signal to said temperature control device coupled to said mattress, said control signal comprising an instruction to turn off.

Embodiment 85. A system for automatically activating a vibrating pillow strip in response to an alarm setting, said system comprising: a database configured to store a history of biological signals associated with a user; said vibrating pillow strip, coupled to a pillow associated with said user, said vibrating pillow strip configured to receive a control signal and to vibrate based on said control signal; a computer processor communicatively coupled to said vibrating pillow strip, and said database, said computer processor configured to: receive a biological signal associated with said user, said biological signal comprising a heart rate associated with said user, a respiration rate associated with said user, a motion associated with said user, or a temperature associated with said user; identify said user based on at least one of: said heart rate associated with said user, said respiration rate associated with said user, said motion associated with said user, or said temperature associated with said user; based on said user identification, obtain a wake-up time associated with said user; based on said history of biological signals and said biological signal, identify a sleep phase associated with said user, said sleep phase comprising a wakefulness phase, a light sleep phase, a deep sleep phase, or a rapid eye movement sleep phase; and when said sleep phase comprises said light sleep phase and a current time is at most one hour before said wake-up time, send said control signal to said vibrating pillow strip, said control signal comprising an instruction to vibrate.

Embodiment 86. The system of embodiment 85, wherein said history of biological signals comprises a normal heart rate range associated with each sleep phase, a normal respiration rate range associated with each sleep phase, a normal motion range associated with each sleep phase, and a normal temperature range associated with each sleep phase.

Embodiment 87. A vibrating alarm system comprising: a vibrating pillow strip, coupled to a pillow associated with a user, said vibrating pillow strip configured to receive a control signal and to vibrate based on said control signal, said vibrating pillow strip comprising: a plurality of vibrating mini motors, wherein each vibrating mini motor is configured to tend to be unnoticeable when said user rests user's head on said vibrating pillow strip, and further wherein said plurality of vibrating mini motors is configured to tend to wake up the user; a computer processor communicatively coupled to said vibrating pillow strip, said computer processor configured to: obtain a wake-up time associated with said user; and at said wake-up time associated with said user, send said control signal to said vibrating pillow strip.

Embodiment 88. The system of embodiment 87, wherein said vibrating mini motor in said plurality of vibrating mini motors is configured to be at most 100 millimeters² in area, and at most 3 mm in thickness.

Embodiment 89. The system of embodiment 87, wherein said vibrating mini motor in said plurality of vibrating mini motors configured to be at least 1 mm away from a neighboring vibrating mini motor.

Embodiment 90. The system of embodiment 87, wherein said vibrating mini motor in said plurality of vibrating mini motors is configured to vibrate at least at 11,000 rotations per minute at 5 volts.

Embodiment 91. The system of embodiment 87, wherein said vibrating pillow strip is coupled to said computer processor via a wireless communication channel.

Embodiment 92. The system of embodiment 87, wherein each vibrating mini motor in said plurality of vibrating mini motors is configured to vibrate synchronously.

Embodiment 93. The system of embodiment 87, said vibrating pillow strip comprising a plurality of zones corresponding to a plurality of users, wherein each zone in said plurality of zones is configured to vibrate independently.

Embodiment 94. The system of embodiment 93, wherein said control signal comprises an identification associated with said zone in said plurality of zones.

Embodiment 95. The system of embodiment 87, said computer processor configured to: receive a biological signal associated with said user, said biological signal comprising a heart rate associated with said user, a respiration rate associated with said user, a motion associated with said user, or a temperature associated with said user; identify said user based on at least one of: said heart rate associated with said user, said respiration rate associated with said user, said motion associated with said user, or said temperature associated with said user; based on said user identification, obtain said wake-up time associated with said user; based on said biological signal, detect when said user is in a light sleep phase; and when said user is in said light sleep phase and a current time is at most one hour before said wake-up time, send said control signal to said vibrating pillow strip, said control signal comprising an instruction to vibrate.

Embodiment 96. The system of embodiment 95, wherein said computer processor is configured to detect when said user is in said light sleep phase by detecting: a slowdown in said heart rate, a drop in said temperature, and a regular respiration rate.

Embodiment 97. The system of embodiment 95, wherein said computer processor is configured to detect when said user is in said light sleep phase by detecting an end of a rapid eye movement sleep phase.

Embodiment 98. The system of embodiment 95, further comprising a database configured to store a history of biological signals associated with said user, wherein said history of biological signals comprises a normal heart rate range associated with each sleep phase, a normal respiration rate range associated with each sleep phase, a normal motion range associated with each sleep phase, and a normal temperature range associated with each sleep phase.

Embodiment 99. The system of embodiment 98, wherein said computer processor is further configured to: based on said user identification, retrieve from said database, said history of biological signals associated with said user; and based on said biological signal and said history of biological signals, determine said sleep phase associated with said user.

Embodiment 100. A method for automatically activating a vibrating pillow strip in response to a biological signal associated with a user, said method comprising: (a) receiving said biological signal associated with said user, said biological signal comprising a heart rate associated with said user, a respiration rate associated with said user, a motion associated with said user, or a temperature associated with said user; (b) retrieving a history of biological signals associated with said user from a database; (c) identifying said user based on at least one of: said heart rate associated with said user, said respiration rate associated with said user, said motion associated with said user, or said temperature associated with said user; (d) based on said user identification, obtaining a wake-up time associated with said user; (e) based on said history of biological signals and said biological signal, identifying a sleep phase associated with said user, said sleep phase comprising a wakefulness phase, a light sleep phase, a deep sleep phase, or a rapid eye movement sleep phase; and (f) when said sleep phase comprises said light sleep phase and a current time is at most one hour before said wake-up time, sending a control signal to said vibrating pillow strip, said control signal comprising an instruction to vibrate.

Embodiment 101. The method of embodiment 100, comprising configuring said vibrating pillow strip to attach to a pillow, to receive said control signal and to vibrate based on said control signal, said vibrating pillow strip comprising a plurality of zones corresponding to a plurality of users, wherein each zone in said plurality of zones is configured to vibrate independently.

Embodiment 102. The method of embodiment 100, wherein said history of biological signals comprises a normal heart rate range associated with each sleep phase, a normal respiration rate range associated with each sleep phase, a normal motion range associated with each sleep phase, and a normal temperature range associated with each sleep phase.

Embodiment 103. A method for automatically activating a vibrating alarm system, said method comprising: (a) configuring a vibrating pillow strip to couple to a pillow associated with a user; (b) configuring said vibrating pillow strip to receive a control signal and to vibrate based on said control signal; (c) coupling a plurality of vibrating mini motors to said vibrating pillow strip, wherein each of vibrating mini motor in said plurality of vibrating mini motors tends to be unnoticeable when a user rests user's head on said vibrating pillow strip, and further wherein said plurality of vibrating mini motors tends to wake up said user; (d) communicatively coupling a computer processor to said vibrating pillow strip; (e) configuring said computer processor to: obtain a wake-up time associated with said user; and at said wake-up time associated with said user, send said control signal to said vibrating pillow strip.

Embodiment 104. The method of embodiment 103, wherein said vibrating mini motor in said plurality of vibrating mini motors is configured to be at most 10 mm in diameter, and at most 2.7 mm in thickness.

Embodiment 105. The method of embodiment 103, wherein said vibrating mini motor in said plurality of vibrating mini motors is configured to vibrate at least at 11,000 rotations per minute at 5 volts.

Embodiment 106. The method of embodiment 103, wherein said vibrating mini motor in said plurality of vibrating mini motors is configured to vibrate synchronously.

Embodiment 107. The system of embodiment 103, wherein said vibrating pillow strip is coupled to said computer processor via a wireless personal area network.

Embodiment 108. The method of embodiment 103, said vibrating pillow strip comprising a plurality of zones corresponding to a plurality of users, wherein each zone in said plurality of zones is configured to vibrate independently.

Embodiment 109. The method of embodiment 108, wherein said control signal comprises an identification associated with said zone in said plurality of zones.

Embodiment 110. The method of embodiment 108, said computer processor configured to: receive a biological signal associated with said user, said biological signal comprising a heart rate associated with said user, a respiration rate associated with said user, a motion associated with said user, or a temperature associated with said user; identify said user based on at least one of: said heart rate associated with said user, said respiration rate associated with said user, said motion associated with said user, or said temperature associated with said user; based on said user identification, obtain said wake-up time associated with said user; based on said biological signal, detect when said user is in a light sleep phase; and when said user is in said light sleep phase and a current time is at most one hour before said wake-up time, send said control signal to said vibrating pillow strip, said control signal comprising an instruction to vibrate.

Embodiment 111. The method of embodiment 110, wherein said computer processor is configured to detect when said user is in said light sleep phase by detecting: a slowdown in said heart rate, a drop in said temperature, and a regular respiration rate.

Embodiment 112. The method of embodiment 110, wherein said computer processor is configured to detect when said user is in said light sleep phase by detecting an end of a rapid eye movement sleep phase.

Embodiment 113. The method of embodiment 110, further comprising a database configured to store a history of biological signals associated with said user, wherein said history of biological signals comprises a normal heart rate range associated with each sleep phase, a normal respiration rate range associated with each sleep phase, a normal motion range associated with each sleep phase, and a normal temperature range associated with each sleep phase.

Embodiment 114. The method of embodiment 113, wherein said computer processor is further configured to: based on said user identification, retrieve from said database, said history of biological signals associated with said user; and based on said biological signal and said history of biological signals, determine said sleep phase associated with said user.

Embodiment 115. A bed system for measuring a biological signal associated with a user and an environment property, and based on said biological signal and said environment property automatically adjusting a temperature of a mattress, said bed system comprising: a bed device configured to receive an instruction to heat said mattress to a specified temperature, said bed device comprising a mattress cover, said mattress cover further comprising: a sensor strip, wherein said sensor strip comprises a user sensor configured to measure said biological signal associated with said user, wherein said biological signal comprises a heart rate associated with said user, and a breathing rate associated with said user; a temperature regulating layer configured to adjust said temperature of said mattress; an environment sensor, configured to measure said environment property, wherein said environment property comprises a current time, a temperature, a humidity, a light intensity, or a sound intensity; a processor communicatively coupled to said bed device and said environment sensor, said processor configured to: determine, based on said biological signal associated with said user and said environment property, a change in a sleep state associated with said user; and when said change in said sleep state comprises a transition from awake to asleep, send a control signal to said bed device.

Embodiment 116. The bed system of embodiment 115, wherein said processor determines presence associated with said user, and motion associated with said user, based on said biological signal associated with said user.

Embodiment 117. The bed system of embodiment 115, wherein said processor is configured to: receive an input from said user; and based on said input from said user, determine at least one of said control signal, or a time to send said control signal to said bed device.

Embodiment 118. The bed system of embodiment 117, wherein said time comprises an average bedtime associated with said user.

Embodiment 119. The bed system of embodiment 115, further comprising: a fabric, a batting, or a waterproof material.

Embodiment 120. The bed system of embodiment 115, wherein said control signal comprises an instruction to heat said temperature regulating layer to a high temperature, wherein said high temperature comprises an average high temperature associated with said user.

Embodiment 121. The bed system of embodiment 115, said processor configured to: based on said biological signal associated with said user, determine said sleep state associated with said user, and a confidence threshold associated with said sleep state; adjust a likelihood that said user is asleep based on said environment property and said confidence threshold associated with said sleep state; and when said likelihood that said user is asleep is above a specified threshold, send said control signal.

Embodiment 122. The bed system of embodiment 121, said control signal comprising an instruction to said bed device to adjust a temperature associated with said bed device to an average temperature associated with said current time and said user.

Embodiment 123. A bed system comprising: a user sensor configured to measure a biological signal associated with a user, said user sensor associated with a bed device; an environment sensor, configured to measure an environment property, wherein said environment property comprises a current time; a beverage-dispensing appliance configured to receive a control signal; a processor communicatively coupled to said user sensor and said environment sensor, and further communicatively coupled to said beverage dispensing appliance, said processor configured to: determine, based on said biological signal associated with said user and said environment property, said control signal, and a time to send said control signal to said beverage dispensing appliance; and send said control signal at said time to said beverage dispensing appliance.

Embodiment 124. The bed system of embodiment 123, wherein said time comprises an average daily wake-up time associated with said user.

Embodiment 125. The bed system of embodiment 123, wherein said beverage dispensing appliance comprises at least one of a coffee maker, a tea maker, a juicer, and a cocktail maker.

Embodiment 126. The bed system of embodiment 123, wherein said time comprises an activation time of an alarm associated with said user.

Embodiment 127. The bed system of embodiment 123, wherein said bed device comprises a coil.

Embodiment 128. The bed system of embodiment 127, further comprising: a fabric, a batting, or a waterproof material.

Embodiment 129. The bed system of embodiment 128, wherein said batting comprises a sensor strip, wherein said sensor strip comprises said user sensor and a computer bus.

Embodiment 130. The bed system of embodiment 123, wherein said biological signal corresponds to at least one of: a presence associated with said user, a motion associated with said user, a temperature associated with said user, a breathing rate associated with said user, or a heart rate associated with said user.

Embodiment 131. The bed system of embodiment 123, wherein said user sensor comprises at least one of a piezo sensor, or a temperature sensor.

Embodiment 132. The bed system of embodiment 123, said processor configured to: determine a sleep state associated with said user, and a confidence threshold associated with said sleep state; adjust a likelihood that said user is asleep based on said environment property and said confidence threshold associated with said sleep state, wherein said environment property comprises at least one of a light intensity, a humidity, and a sound intensity; and based on said likelihood, send said control signal to said beverage dispensing appliance.

Embodiment 133. The bed system of embodiment 123, wherein said processor is configured to: receive input from said user; and based on said input from said user, determine said time to send said control signal to said beverage dispensing appliance.

Embodiment 134. The bed system of embodiment 133, wherein said processor configured to determine said time comprises said processor configured to: record a wake-up time when a presence associated with said user is no longer detected; upon recording said wake-up time, add said input from said user to said wake-up time to obtain said time at which to activate said beverage dispensing appliance.

Embodiment 135. A system comprising: a user sensor configured to measure a biological signal associated with a user, said biological signal comprising at least one of a temperature associated with said user, a presence associated with said user, a motion associated with said user, a breathing rate associated with said user, and a heart rate associated with said user; an environment sensor, configured to measure an environment property; a transceiver communicatively coupled to said user sensor and said environment sensor, said transceiver configured to transmit said biological signal associated with said user and said environment property, and further said transceiver configured to receive a control signal; a bed device, communicatively coupled to said transceiver, wherein said bed device is configured to respond to said control signal; and a processor, communicatively coupled to said transceiver, wherein said processor is configured to identify said user to obtain an identified user.

Embodiment 136. The system of embodiment 135, wherein said environment property comprises at least one of a temperature, a humidity, a light intensity, and a sound intensity.

Embodiment 137. The system of embodiment 135, wherein said control signal comprises an instruction to heat said bed device to a high temperature, wherein said high temperature comprises an average high temperature associated with said identified user.

Embodiment 138. The system of embodiment 135, wherein said control signal comprises an instruction to cool said bed device to a low temperature, wherein said low temperature comprises an average low temperature associated with said identified user.

Embodiment 139. A method comprising: (a) obtaining a biological signal associated with a user from a sensor, wherein said biological signal comprises at least one of a presence associated with said user, a motion associated with said user, a breathing rate associated with said user, a temperature associated with said user, and a heart rate associated with said user; (b) identifying said user to obtain a user ID, said identifying based on at least one of said heart rate associated with said user, said breathing rate associated with said user, said temperature associated with said user, or said motion associated with said user; (c) based on said user ID, obtaining a history of biological signals associated with said user ID from a database; (d) obtaining an environment property from an environment sensor associated with a bed device; (e) obtaining an average associated with said environment property from said database; (f) determining, based on said biological signal associated with said user, said history of biological signals associated with said user ID, said environment property, and said average associated with said environment property a control signal, and a time to send said control signal to said bed device; and (g) sending said control signal at said time to said bed device.

Embodiment 140. The method of embodiment 139, wherein obtaining said history of biological signals comprises: obtaining a plurality of average bedtimes associated with said user ID, wherein said plurality of average bedtimes comprises an average bedtime on one or more days of a week.

Embodiment 141. The method of embodiment 139, wherein sending said control signal comprises: sending an instruction to heat said bed device to a high temperature, wherein said high temperature comprises an average high temperature associated with said user ID.

Embodiment 142. The method of embodiment 139, wherein sending said control signal comprises: sending an instruction to cool said bed device to a low temperature, wherein said low temperature comprises an average low temperature associated with said user ID.

Embodiment 143. The method of embodiment 139, wherein obtaining said environment property comprises: obtaining at least one of a temperature, a humidity, a light intensity, and a sound intensity.

Embodiment 144. The method of embodiment 139, wherein determining said time to send said control signal to said bed device comprises obtaining an average daily bedtime associated with said user ID.

Embodiment 145. A system for automatically activating a vibrating alarm strip in response to a biological signal associated with a user, said system comprising: a sensor strip configured to measure said biological signal associated with said user, wherein said sensor strip comprises a piezo sensor, and a temperature sensor, and further wherein said biological signal comprises a breathing rate associated with said user, a heart rate associated with said user, a motion associated with said user, and a temperature associated with said user; a database configured to store a history of biological signals associated with said user; said vibrating alarm strip, attached to a mattress, configured to receive a control signal and to vibrate based on said control signal, said vibrating alarm strip comprising a plurality of zones corresponding to a plurality of users, wherein each zone in said plurality of zones is configured to vibrate independently; a computer processor communicatively coupled to said sensor strip, said vibrating alarm strip, and said database, said computer processor configured to: identify said user based on at least one of: said heart rate associated with said user, said breathing rate associated with said user, said motion associated with said user, or said temperature associated with said user; based on said user identification, obtain a wake-up time associated with said user; based on said history of biological signals and said biological signal, identify a sleep phase associated with said user, said sleep phase comprising a wakefulness phase, a light sleep phase, a deep sleep phase, or a rapid eye movement sleep phase; and when said sleep phase comprises said light sleep phase and a current time is at most one hour before said wake-up time, send said control signal to said vibrating alarm strip, said control signal comprising an instruction to vibrate.

Embodiment 146. The system of embodiment 145, wherein said history of biological signals comprises a normal heart rate range associated with each sleep phase, a normal breathing rate range associated with each sleep phase, a normal motion range associated with each sleep phase, and a normal temperature range associated with each sleep phase.

Embodiment 147. A system for automatically activating a vibrating alarm in response to a biological signal associated with a user, said system comprising: a sensor strip configured to measure said biological signal associated with said user, wherein said sensor strip comprises a piezo sensor, and a temperature sensor, and further wherein said biological signal comprises a breathing rate associated with said user, a heart rate associated with said user, a motion associated with said user, and a temperature associated with said user; said vibrating alarm, coupled to said user, configured to receive a control signal and to vibrate based on said control signal; a computer processor communicatively coupled to said sensor strip, and said vibrating alarm, said computer processor configured to: identify said user based on at least one of: said heart rate associated with said user, said breathing rate associated with said user, said motion associated with said user, or said temperature associated with said user; based on said user identification, obtain a wake-up time associated with said user; based on said biological signal, detect when said user is in a light sleep phase; and when said user is in said light sleep phase and a current time is at most one hour before said wake-up time, send said control signal to said vibrating alarm, said control signal comprising an instruction to vibrate.

Embodiment 148. The system of embodiment 147, wherein said computer processor is configured to detect when said user is in said light sleep phase by detecting: a slowdown in said heart rate, a drop in said temperature, and a regular breathing rate.

Embodiment 149. The system of embodiment 147, wherein said computer processor is configured to detect when said user is in said light sleep phase by detecting an end of said rapid eye movement sleep phase.

Embodiment 150. The system of embodiment 147, said vibrating alarm comprising a vibrating alarm strip, said vibrating alarm strip coupled to said user, and further said vibrating alarm strip comprising a plurality of zones corresponding to a plurality of users, wherein each zone in said plurality of zones is configured to vibrate independently.

Embodiment 151. The system of embodiment 150, wherein said control signal comprises an identification associated with said zone in said plurality of zones.

Embodiment 152. The system of embodiment 147, said vibrating alarm comprising a vibrating alarm strip attached to a bed pad.

Embodiment 153. The system of embodiment 147, said vibrating alarm comprising a vibrating alarm strip attached to a bed cover.

Embodiment 154. The system of embodiment 147, said vibrating alarm comprising a vibrating alarm strip attached to a bed sheet.

Embodiment 155. The system of embodiment 147, said vibrating alarm comprising a wearable user device.

Embodiment 156. The system of embodiment 147, further comprising a database configured to store a history of biological signals associated with said user, wherein said history of biological signals comprises a normal heart rate range associated with each sleep phase, a normal breathing rate range associated with each sleep phase, a normal motion range associated with each sleep phase, and a normal temperature range associated with each sleep phase.

Embodiment 157. The system of embodiment 156, wherein said computer processor is further configured to: based on said user identification, retrieve from said database, said history of biological signals associated with said user; and based on said biological signal and said history of biological signals, determine said sleep phase associated with said user.

Embodiment 158. A method for automatically activating a vibrating alarm strip in response to a biological signal associated with a user, said method comprising: (a) measuring said biological signal associated with said user with a sensor strip, wherein said sensor strip comprises a piezo sensor, and a temperature sensor, and further wherein said biological signal comprises a breathing rate associated with said user, a heart rate associated with said user, a motion associated with said user, and a temperature associated with said user; (b) storing a history of biological signals associated with said user in a database; (c) identifying said user based on at least one of: said heart rate associated with said user, said breathing rate associated with said user, said motion associated with said user, or said temperature associated with said user; (d) based on said user identification, obtaining a wake-up time associated with said user; (e) based on said history of biological signals and said biological signal, identifying a sleep phase associated with said user, said sleep phase comprising a wakefulness phase, a light sleep phase, a deep sleep phase, or a rapid eye movement sleep phase; and (f) when said sleep phase comprises said light sleep phase and a current time is at most one hour before said wake-up time, sending a control signal to said vibrating alarm strip, said control signal comprising an instruction to vibrate.

Embodiment 159. The method of embodiment 158, comprising configuring said vibrating alarm strip to attach to a mattress, to receive said control signal and to vibrate based on said control signal, said vibrating alarm strip comprising a plurality of zones corresponding to a plurality of users, wherein each zone in said plurality of zones is configured to vibrate independently.

Embodiment 160. The method of embodiment 158, wherein said history of biological signals comprises a normal heart rate range associated with each sleep phase, a normal breathing rate range associated with each sleep phase, a normal motion range associated with each sleep phase, and a normal temperature range associated with each sleep phase.

Embodiment 161. A method for automatically activating a vibrating alarm in response to a biological signal associated with a user, said method comprising: (a) configuring a computer processor to: identify said user based on at least one of: a heart rate associated with said user, a breathing rate associated with said user, a motion associated with said user, or a temperature associated with said user, wherein said biological signal comprises said heart rate, said breathing rate, said motion, and said temperature; (b) based on said user identification, obtain a wake-up time associated with said user; (c) based on said biological signal, detect when said user is in a light sleep phase; and (d) when said user is in said light sleep phase and a current time is at most one hour before said wake-up time, send a control signal to said vibrating alarm coupled to said user, said control signal comprising an instruction to vibrate.

Embodiment 162. The method of embodiment 161, further comprising: configuring a sensor strip to measure said biological signal associated with said user, wherein said sensor strip comprises a piezo sensor, and a temperature sensor.

Embodiment 163. The method of embodiment 161, further comprising: configuring said vibrating alarm to receive said control signal and to vibrate based on said control signal.

Embodiment 164. The method of embodiment 163, said vibrating alarm comprising a vibrating alarm strip attached to a bed pad.

Embodiment 165. The method of embodiment 163, said vibrating alarm comprising a vibrating alarm strip attached to a bed cover.

Embodiment 166. The method of embodiment 163, said vibrating alarm comprising a vibrating alarm strip attached to a bed sheet.

Embodiment 167. The method of embodiment 163, said vibrating alarm comprising a wearable user device.

Embodiment 168. The method of embodiment 163, wherein said vibrating alarm comprises a vibrating alarm strip coupled to said user, and further wherein said vibrating alarm strip comprises a plurality of zones corresponding to a plurality of users, wherein each zone in said plurality of zones is configured to vibrate independently.

Embodiment 169. The method of embodiment 168, wherein said control signal comprises an identification associated with said zone in said plurality of zones.

Embodiment 170. The method of embodiment 163, wherein said configuring said computer processor to detect when said user is in said light sleep phase comprises detecting a slowdown in said heart rate, a drop in said temperature, and a regular breathing rate.

Embodiment 171. The method of embodiment 163, wherein said configuring said computer processor to detect when said user is in said light sleep phase comprises detecting an end of said rapid eye movement sleep phase.

Embodiment 172. The method of embodiment 163, further comprising configuring a database to store a history of biological signals associated with said user, wherein said history of biological signals comprises a normal heart rate range associated with each sleep phase, a normal breathing rate range associated with each sleep phase, a normal motion range associated with each sleep phase, and a normal temperature range associated with each sleep phase.

Embodiment 173. The method of embodiment 172, further comprising: configuring said computer processor to, based on said user identification, retrieve from said database, said history of biological signals; and configuring said computer processor to, based on said biological signal and said history of said biological signals, determine said sleep phase associated with said user.

What is claimed is:

1. A system comprising:
(a) a bed device comprising:
(i) a mattress or a mattress cover, wherein the mattress or the mattress cover comprises a top surface and a side surface;
(ii) a sensor disposed within the top surface, wherein the sensor is configured to:
(A) detect a biological signal of a user while the user is on the bed device, and
(B) generate an analog biological signal data based on the detection;
(iii) an analog-to-digital converter (ADC) disposed within the side surface and configured to convert the analog biological signal data into a digital biological signal data; and
(iv) a temperature control device disposed adjacent to the top surface and configured to control a temperature of a zone of the bed device; and
(b) a processor separate and remote from the bed device, wherein the processor is configured to receive the digital biological signal data and based on the digital biological signal data direct the temperature control device to change the temperature of the zone of the bed device.

2. The system of claim 1, wherein the sensor and the ADC are provided as components of a layer of the bed device.

3. The system of claim 1, wherein the sensor and the ADC are provided as components of a sensor strip, and the sensor strip is a layer of the bed device.

4. The system of claim 1, wherein the sensor and the ADC are electrically coupled via a wire.

5. The system of claim 1, further comprising a computer bus disposed within the bed device, wherein the computer bus is configured to send the digital biological signal data to the processor.

6. The system of claim 1, wherein the sensor comprises a plurality of sensors for detecting a plurality of types of biological signals.

7. The system of claim 1, wherein the biological signal comprises one or more members selected from the group consisting of a temperature signal associated with the user, a movement signal associated with the user, a presence signal associated with the user, a heart signal associated with the user, and a breathing signal associated with the user.

8. The system of claim 1, wherein the sensor comprises a temperature sensor or a piezo sensor.

9. The system of claim 1, wherein the processor is configured to (i) determine a current sleep phase of the user based on the digital biological signal data, and (ii) cause the temperature control device to change the temperature of the zone of the bed device based on the sleep phase.

10. The system of claim 1, wherein the ADC is in data communication with the sensor via one or more wires that are disposed adjacent to at least a portion of the top surface and at least a portion of the surface side of the mattress or the mattress cover.

11. A method comprising:
(a) providing a bed device comprising:
(i) a mattress or a mattress cover, wherein the mattress or the mattress cover comprises a top surface and a side surface;
(ii) a sensor disposed within the top surface, wherein the sensor is configured to:
(A) detect a biological signal of a user while the user is on the bed device, and
(B) generate an analog biological signal data based on the detection;
(iii) an analog-to-digital converter (ADC) disposed within the side surface and configured to convert the analog biological signal data into a digital biological signal data; and
(iv) a temperature control device disposed adjacent to the top surface and configured to control a temperature of a zone of the bed device;
(b) using the sensor to detect the biological signal of the user and generate the analog biological signal data based on the detection;
(c) using the ADC to convert the analog biological signal data into the digital biological signal data;
(d) sensing the digital biological signal data to a processor, wherein the processor is configured to receive the digital biological signal data, wherein the processor is separate and remote from the bed device; and
(e) using the processor to direct the temperature control device to change the temperature of the zone of the bed device.

12. The method of claim 11, wherein the sensor and the ADC are provided as components of a layer of the bed device.

13. The method of claim 11, wherein the sensor and the ADC are provided as components of a sensor strip, and the sensor strip is a layer of the bed device.

14. The method of claim 11, wherein the sensor and the ADC are electrically coupled via a wire.

15. The method of claim 11, comprising using a computer bus disposed within the bed device to send the digital biological signal data to the processor.

16. The method of claim 11, wherein the sensor comprises a plurality of sensors for detecting a plurality of types of biological signals.

17. The method of claim 11, wherein the biological signal comprises one or more members selected from the group consisting of a temperature signal associated with the user, a movement signal associated with the user, a presence signal associated with the user, a heart signal associated with the user, and a breathing signal associated with the user.

18. The method of claim 11, wherein the sensor comprises a temperature sensor or a piezo sensor.

19. The method of claim 11, comprising (i) determining a current sleep phase of the user based on the digital biological signal data, and (ii) causing the temperature control device to change the temperature of the zone of the bed device based on the sleep phase.

20. The method of claim 11, wherein the ADC is in data communication with the sensor via one or more wires that are disposed adjacent to at least a portion of the top surface and at least a portion of the surface side of the mattress or the mattress cover.

21. A system comprising:
(a) a bed device comprising:
(i) a mattress or a mattress cover, wherein the mattress or the mattress cover comprises a top surface and a side surface;
(ii) a sensor disposed within the top surface, wherein the sensor is configured to:
(A) detect a biological signal of a user while the user is on the bed device, and
(B) generate an analog biological signal data based on the detection;
(iii) an analog-to-digital converter (ADC) disposed within the side surface and configured to convert the analog biological signal data into a digital biological signal data; and
(iv) a temperature control device disposed adjacent to the top surface and configured to control a temperature of a zone of the bed device; and
(b) a processor separate and remote from the bed device, wherein the processor is configured to receive the digital biological signal data and based on the digital biological signal data direct the temperature control device to change the temperature of the zone of the bed device, wherein the processor is configured to (i) determine a current sleep phase of the user based on the digital biological signal data, and (ii) cause the temperature control device to change the temperature of the zone of the bed device based on the current sleep phase to improve a sleep environment of the user.

22. A method comprising:
(a) providing a bed device comprising:
(i) a mattress or a mattress cover, wherein the mattress or the mattress cover comprises a top surface and a side surface;
(ii) a sensor disposed within the top surface, wherein the sensor is configured to:
(A) detect a biological signal of a user while the user is on the bed device, and
(B) generate an analog biological signal data based on the detection;
(iii) an analog-to-digital converter (ADC) disposed within the side surface and configured to convert the analog biological signal data into a digital biological signal data; and
(iv) a temperature control device disposed adjacent to the top surface and configured to control a temperature of a zone of the bed device;
(b) using the sensor to detect the biological signal of the user and generate the analog biological signal data based on the detection;
(c) using the ADC to convert the analog biological signal data into the digital biological signal data;
(d) sending the digital biological signal data to a processor, wherein the processor is configured to receive the digital biological signal data, wherein the processor is separate and remote from the bed device;
(e) using the processor to determine a current sleep phase of the user based on the digital biological signal data; and
(f) using the processor to direct the temperature control device to change the temperature of the zone of the bed device.
